(19) United States
(12) Patent Application Publication
(10) Pub. No.: US 2005/0004007 A1
(43) Pub. Date: Jan. 6, 2005

(54) Promotion of adoptosis in cancer cells by co-administration of cyclin dependent kinase inhibitiors and cellular differentiation agents

(76) Inventors: Steven Grant, Richmond, VA (US); Paul Dent, Glen Allen, VA (US); Roberto Rosato, Richmond, VA (US); Leanne Cartee, Atlanta, GA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/363,540

(22) PCT Filed: Sep. 7, 2001
(86) PCT No.: PCT/US01/28297

(30) Foreign Application Priority Data
Sep. 12, 2000 (US) 60231885

(51) Int. Cl. A61K 38/12; A61K 31/353; A61K 31/19

(52) U.S. Cl. 514/7; 514/9; 514/167; 514/460; 514/456; 514/559; 514/575

(57) ABSTRACT

The invention provides compositions and methods for promoting apoptosis of cancer cells, and methods for treating cancer. The compositions comprise cyclin dependent kinase inhibitor and an agent that induces cellular differentiation. The methods of promoting apoptosis of cancer cells involve the co-administration to the cancer cells of a cyclin dependent kinase inhibitor and an agent that induces cell differentiation. The method for treating cancer involves the co-administration of a cyclin dependent kinase inhibitor and an agent that induces cellular differentiation to a patient. Examples of cyclin dependent kinase inhibitors include histone deacetylase inhibitors, protein kinase C activators, retinoids, and Vitamin D3.

### BACKGROUND OF THE INVENTION

1. Field of the Invention

The invention generally relates to the promotion of apoptosis in cancer cells. In particular, the invention provides methods to promote apoptosis in cancer cells by the co-administration of cyclin dependent kinase inhibitors and agents that induce cellular differentiation.

2. Background of the Invention

Despite the many advances that have been made in the treatment of cancer, there are still many forms of this disease that remain recalcitrant to known treatment regimes. There is thus an ongoing need for the development of new approaches to treatment of the disease.

A hallmark of cancer cells is unrestricted growth of the malignant cells, due, in part, to circumvention of normal cell cycle progression. Normal eukaryotic cell cycle progression is governed by the sequential activation and inactivation of various cyclin/cyclin-dependent kinase (CDK) complexes. Levels of the cyclin proteins (e.g., cyclins D, E, A, and B) fluctuate temporally during cell cycle traverse through G₁, S, G₂ and M [Johnson and Walker, 1999], leading to activation of their respective CDKs (CDK4/6, CDK2, and CDK1) at the appropriate time. A major function of the cyclin D/CDK 4 complex is to phosphorylate the retinoblastoma protein (pRb) at or near the restriction checkpoint of G₀/G_{1.} This, in turn, promotes cellular proliferation and S-phase progression through liberation of the E2F transcription factor normally bound to hypophosphorylated pRb [Pines 1995]. Free E2F triggers the expression of diverse genes related to S-phase progression, including dihydrofolate reductase and thymidylate synthetase [Fan and Bertino, 1997]. Conversely, cyclin-dependent kinase inhibitors (CDKIs) such as p21^{WAF1/CIP1} and p27^{KIP1} play an important role in cell cycle control by coordinating internal and external signals that impede cell cycle progression at key checkpoints [Sherr and Roberts, 1995]. When the CDKIs p21^{WAF1/CIP1} and p27^{KIP1} are induced, CDKs are inhibited, resulting in pRb dephosphorylation [Weinberg, 1995]. Hypophosphorylated pRb, in turn, binds to and inactivates E2F, thereby promoting cell cycle arrest in G₁.

Recently, attention has focused on the development of pharmacologic inhibitors of CDKs as anticancer agents [Christian et al, 1997]. Flavopiridol (L86-8275; NCS 649890; FP) is a CDK inhibitor that is currently undergoing Phase I/II clinical trials in humans [Senderowicz et al., 1998]. FP interacts with the adenine-binding pocket of CDKs to inhibit kinase activity at concentrations ˜100 nM for CDKs 1, 2, 4, and 6, and 300 nM for CDK 7, the CDK-activating kinase [Senderowicz et al., 1999]. In addition, FP decreases the expression of cyclins D1, D3, and E without modifying cyclin D2 protein levels [Carlson et al., 1999]. Concordant with its ability to block the expression of various cyclins and inhibit CDK activity, FP induces either G₁ and/or G₂/M cell cycle arrest. Furthermore, FP is a potent inducer of apoptosis in human leukemia cells [Parker et al., 1998], and also potentiates the lethality of conventional cytotoxic agents in a sequence-dependent manner [Bible and Kaufmann, 1997]. Although the mechanism by which FP promotes apoptosis in leukemia cells remains unknown, it is presumed that FP-mediated cytotoxicity stems from cell cycle perturbations [Lundberg and Weinberg, 1999], particularly in view of abundant evidence that disruption of cell cycle progression represents a potent apoptotic stimulus [Meikrantz and Schlegel, 1995].

Aside from studies involving cytotoxic agents, little information is currently available concerning interactions between FP and other classes of drugs for use in the treatment of cancer. One potential class for investigation is that of agents that induce differentiation or maturation of cells. The induction of differentiation in tumor cells diverts them from uncontrolled growth to a normal cell cycle.

One example of an agent that is known to induce cell differentiation is phorbol 12-myristate 13-acetate (PMA). PMA is a protein kinase C (PKC) activator and tumor promoter that induces terminal differentiation (maturation) in human leukemia cells [Jiang et al. 1996]. Leukemic cell maturation triggered by PMA, or other differentiation-inducers, requires exit from the cell cycle and G₁ arrest [Carey et al. 1996]. The cell cycle arrest induced by PMA is preceded by increased expression of the CDKIs p21^{WAF1/CIP1} and p27^{KIP1} in a p53-independent manner [Jiang et al. 1996]. The role of CDKIs in leukemic cell maturation has been underscored by the observation that stable expression of a p21^{WAF1/CIP1} antisense construct in U937 monocytic leukemia cells blocks PMA-induced differentiation and reciprocally promotes apoptosis in these cells [Wang et al. 1998]. Conversely, enforced expression of CDKIs has been linked to cytotoxic drug resistance and protection from apoptosis [Ruan et al, 1998; Eymin, et al. 1999]. Furthermore, it has been reported that p21^{WAF1/CIP1} can form a complex with procaspase-3 to inhibit Fas-mediated apoptosis, raising the possibility of a direct role for this CDKI in preventing cell death [Suzuki et al. 1998]. Taken together, these findings provide evidence that CDKIs such as p21^{WAF1/CIP1} oppose apoptosis, and suggest a possible mechanism by which induction of leukemic cell maturation may, under some circumstances, protect cells from genotoxic stresses [Sordet et al, 1999].

A second example of a differentiation inducing agent is the histone deacetylase inhibitor sodium butyrate (SB). SB is a non-toxic short chain fatty acid found naturally in the gastrointestinal tract that appears to be responsible for the protective effects attributed to high-fiber diets (Trock et al., 1990; McIntyre et al., 1991, 1993). In this regard, SB has been shown to exhibit activity in a rat model of colon cancer (McIntyre et al., 1993), to suppress proliferation of cancer cell lines (Pellizaro et al., 2001) and to increase markers of cell differentiation (Rosato et al., 2001). Like other histone deacetylase inhibitors (HDIs), it promotes acetylation of histones, leading to alterations in chromosomal structure that promote expression of genes involved in cellular maturation. It is also able to induce tumor cell apoptosis in a dose-dependent manner. From a functional standpoint, SB increases the expression of the cyclin-dependent kinase inhibitor (CDKI) p21^{WAF1/CIP1/MDA6}, which leads in turn to growth arrest in the G₁ phase of the cell cycle. p21^{WAF1/CIP1} is known to play an important role in promoting differentiation and preventing mithocondrial dysfunction and apoptosis in human leukemia cells (i.e., HL-60 and U937) (Freemerman, 1997). For example, U937 cells stably transfected with a p21^{WAF1/CIP1} antisense construct were significantly more susceptible to ara-C— and SB-mediated mitochondrial injury and apoptosis than their wild-type counterparts (Wang et al. 32, Rosato et al., 2001).

Given the known requirement for G₁ arrest in leukemic cell differentiation programs [Carey et al. 1996], the capacity of a cyclin dependent kinase inhibitor to block cell cycle progression would be expected to lower the differentiation threshold, i.e. to facilitate the induction of differentiation in cells. To test this hypothesis, the effects of cyclin dependent kinase inhibitors on the activity of several differentiation inducing agents was assayed by co-administering the two types of compounds. Surprisingly, the cyclin dependent kinase inhibitors did not cause an increase in the ability of the differentiation agents to initiate cellular differentiation. Rather, the co-administration of the two types of compounds unpredictably resulted in elevated levels of apoptosis of the targeted cancer cells.

### SUMMARY OF THE INVENTION

It is an object of the present invention to provide a method for promoting apoptosis in cancer cells. The method involves co-administering to the cancer cells to a cyclin dependent kinase inhibitor and an agent that induces cellular differentiation. Several categories of agents that induce cellular differentiation may be utilized in the invention, including histone deacetylase inhibitors [such as sodium butyrate, phenylbutyrate, suberoylanilide hydroxamic acid (SAHA), depsipeptide, tricostatin A, MS-275, and CI-994], protein kinase C activators [such as PMA and bryostatin], retinoids [such as all trans retinoic acid (ATRA)], and Vitamin D3. Cyclin dependent kinase inhibitors that may be used in the practice of the invention include flavopiridol, UCN-01, roscovitine, olomoucine, and butyrolactone. Examples of types of cancer cells in which apoptosis may be promoted by the invention are leukemia cells, prostate cancer cells, breast cancer cells, multiple myeloma cells, and lymphoma cells.

It is a further object of the present invention to provide a method for the treatment of cancer in a patient in need thereof by co-administering to the patient a cyclin dependent kinase inhibitor and an agent that induces cellular differentiation. Several categories of agents that induce cellular differentiation may be utilized in this aspect of the invention, including histone deacetylase inhibitors [such as sodium butyrate, phenylbutyrate, suberoylanilide hydroxamic acid (SAHA), depsipeptide, tricostatin A, MS-275, and CI-994], protein kinase C activators [such as PMA and bryostatin], retinoids [such as all trans retinoic acid (ATRA)]), and Vitamin D3. Cyclin dependent kinase inhibitors that may be used in the practice of the invention include flavopiridol, UCN-01, roscovitine, olomoucine, and butyrolactone. Examples of types of cancer that may be treated by the promotion of apoptosis by the method of the present invention include leukemia, prostate cancer, breast cancer, multiple myeloma cells, and lymphoma cells.

In yet another aspect of the present invention, a novel composition of matter is provided. The composition of matter comprises a cyclin dependent kinase inhibitor, an agent that induces cellular differentiation, and a carrier suitable for in vivo administration. Several categories of agents that induce cellular differentiation may be utilized in this aspect of the invention, including histone deacetylase inhibitors [such as sodium butyrate, phenylbutyrate, suberoylanilide hydroxamic acid (SAHA), depsipeptide, tricostatin A, MS-275, and CI-994], protein kinase C activators [such as PMA and bryostatin], retinoids [such as all trans retinoic acid (ATRA)]), and Vitamin D3. Cyclin dependent kinase inhibitors that may be used in this aspect of the invention include flavopiridol, UCN-01, roscovitine, olomoucine, and butyrolactone.

### BRIEF DESCRIPTION OF THE DRAWINGS

FIG. 1A-D. A, The percentage of apoptotic cells induced by varying concentrations of FP (50-400 nM) and drug exposure intervals (6-24 h) in U937 human leukemia cells. Values represent the means for three separate experiments±standard error. B, The percentage of apoptotic cells induced by 10 nM PMA×24 h, 100 nM FP×24 h, or co-incubation (10 nM PMA/100 nM FP×24 h) versus sequential exposure schedules (10 nM PMA×24 h [symbol] 100 nM FP×24 h or 100 nM FP [symbol] 10 nM PMA×24 h) of these agents in U937 cells. Values represent the means for four separate experiments±standard error. C, The percentage of apoptotic cells induced by 10 nM PMA×24 h, 100 nM FP×24 h, 10 nM PMA/100 nM FP×24 h or 1.0 μM ara-C×6 h in empty-vector control (U937/pCEP4) and Bcl-2 overexpressing cells (U937/Bcl-2); and D, HL60 human promyelocytic leukemia cells exposed to 10 nM PMA±200 nM FP. Values represent the means for three separate experiments±standard error (C-D). Apoptotic cells were identified by morphologic analysis of Wright and Giemsa-stained cytospin preparations as described in the text (A-D).

FIG. 2A-B. A, The percentage of apoptotic cells induced by 24-h exposures to PMA (5, 8, 10 nM), FP (50, 80, or 100 nM), or the combination of PMA and FP in U937 cells. Values represent the means for seven separate experiments±standard error. Drug combinations were administered in a 1:10 constant ratio and interactions were analyzed by Median Dose Effect analysis. A C.I. value <0.01 denotes significant synergism. Apoptotic cells were identified by morphologic analysis of Wright and Giemsa-stained cytospin preparations as described in the text. B, Clonogenic survival of U937 cells following 24-h exposures to 10 nM PMA, 50 or 100 nM FP, and 10 nM PMA/50 or 100 nM FP co-treatments. Values represent the means for triplicate determinations from three separate experiments±standard error, and are expressed as a percentage relative to untreated controls. PMA combined with 100 nM FP significantly reduced clonogenic survival of U937 cells compared to PMA alone (*p<0.03).

FIG. 3. The loss of mitochondrial membrane potential (Δψₘ) was monitored over an 8-h time course in U937 cells exposed to 10 nM PMA, 100 nM FP, or 10 nM PMA/100 nM FP co-treatment±20 μM B-D-FMK. Values represent the means for three separate experiments±standard error and are expressed as the percentage of cells expressing low mitochondrial membrane potential (Δψₘ), manifested by reduced levels of DiOC₆ uptake relative to untreated controls.

FIG. 4A-B. A, The percentage of U937 cells expressing the CD11b surface differentiation marker following 24-h exposures to 10 nM PMA, 50 nM FP, or to the combination of 10 nM PMA and 50 nM FP relative to untreated controls. Values represent the means of four separate experiments±standard error. FP co-treatment significantly reduced CD11b expression compared to PMA alone (*p<0.03) B, The percentage of adherent U937 cells following treatment with 10 nM PMA, 100 nM FP and 10 nMPMA/100 nM FP. Values represent the means of three separate experiments±standard error. FP co-treatment profoundly decreased the plastic adherence of cells exposed to PMA (#p<0.001).

FIG. 5A-B. A, Time course study of apoptosis induction by 10 nM PMA or 10 nM PMA/100 nM FP in the presence or absence of 20 μM B-D-FMK, a pancaspase inhibitor (10P/100F+I). Apoptotic cells were identified by morphologic analysis as described in the text. Values represent the means of three separate experiments±standard error. B, Transient transfection of U937 cells with a p21^{WAF1/CIP1} luciferase reporter construct containing the full-length promoter. U937 cells were treated for 3 or 6 h with 10 nM PMA, 100 nM FP or 10 nM PMA/100 nM FP co-treatment and reporter activity was determined by luminometry. Values represent mean RLU normalized to mU of β-galactosidase activity relative to untreated controls for three separate assays, and generally did not vary >20% for individual experiments.

FIG. 6A-B. A, Effects of the specific MEK1 inhibitors, PD98059 (25 μM; 25 PD) or U0126 (25 μM; 25 U6), on MAPK phosphorylation (p44/p42) and activation were determined by analysis of U937 cell lysates obtained after 1 h exposures to 10 nM PMA (P) or 10 nM/100 nM FP co-treatment (P/F) as described in Methods. The percentage of apoptotic cells was determined by morphologic analysis of Wright and Giemsa-stained cytospin preparations as described in the text. The addition of 25 μM U0126 did not significantly attentuate the apoptotic response of U937 cells to 10 nM PMA/100 nM FP co-treatment. Values represent the means of three separate experiments±standard deviation. B, The percentage of apoptotic cells induced by 24-h exposures to PMA (1.0 nM), FP (50 and 80 nM), and the combination of PMA and FP was determined in U937/pREP4 (open bars) and U937/p21AS cells (closed bars) as outlined in Methods. Values represent the means of three separate experiments±standard error. Apoptotic cells were identified by morphologic analysis of Wright and Giemsa-stained cytospin preparations as described in the text.

FIGS. 7A and B.: FP/SB co-treatment works as a potent apoptosis inducer combination. A, percentage of apoptosis observed in U937 myelomonocytic human leukemia cells exposed to 1 mM SB, 100 nM FP or the combination of both compounds during 24 h and 48 h. Apoptotic cells were identified by morphological analysis of Wright and Giemsa-stained cytospin preparations as described in the text. B, percentage of FP/SB-induced apoptosis in U937, HL-60 promyelocytic, Jurkat T-lymphoblastic and Raji B-lymphoblastic cell lines. In both A and B C=control, B=butyrate alone, F=flavopiridol alone, and FB=the combination of butyrate and flavopiridol.

FIGS. 8A and B: A, U937 cells were treated 12 hours with FP, SB or the combination of both in absence or presence of either 20 μM pan-caspase inhibitor BOC-D-fmk or 20 μM caspase-8 inhibitor Z-IEDT-fmk. Apoptosis was measured by morphological analysis of Wright and Giemsa-stained cytospin preparations. B, the loss of mitochrondrial membrane potential (Δψₘ) was monitored at different time points in cells exposed to 100 nM FP, 1 mM SB or the combination of both. Values represent the means for three separate experiments±SE an are expressed as the percentage of cells expressing low mitochondrial membrane potential (Δψₘ) as manifested by reduced levels of DioC₆ uptake relative to untreated controls.

FIGS. 9A and B: A, cell cycle analysis of cells after exposure to FP/SB alone or in combination. U937 cells were incubated in 70% ethanol and then treated with propidium iodide and subjected to cell cycle analysis using a Becton Dickinson FACScan flow cytometer and ModFit software, as described in MM. The results of a representative study are shown; additional experiments yielded similar results. B, FP/SB-treated U937 cells failed to up-regulate CD11b expression in response to SB. U937 cells were incubated with 100 nM FP, 1 mM SB or the combination of both for 48 h and the percentage of cells expressing the CD11b surface differentiation marker determined by flow cytometry. Values represent the means±S.D. for three separate experiments.

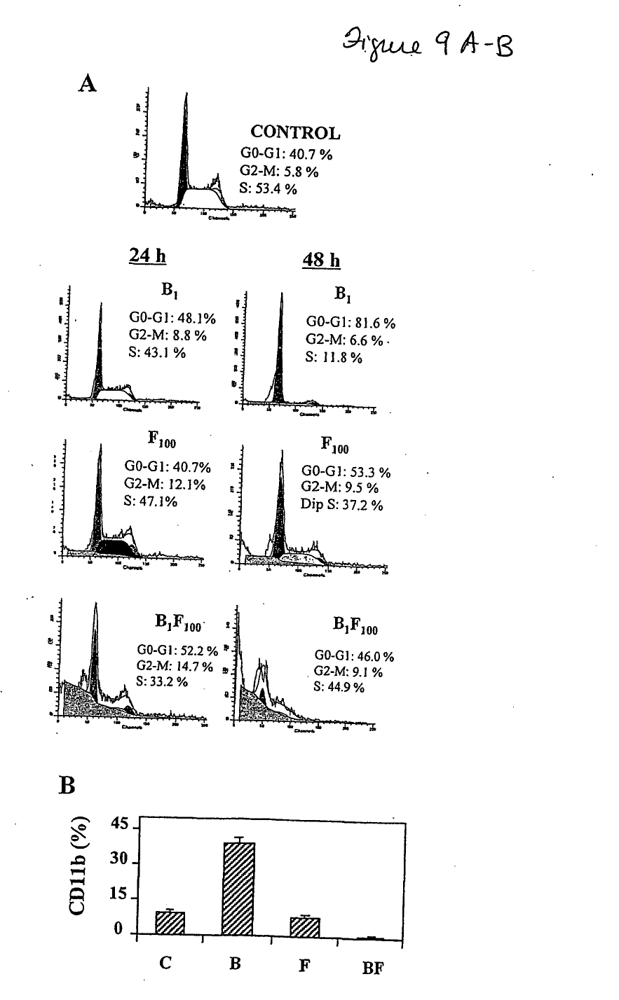

FIG. 10: Percentage of apoptosis observed in blasts isolated from two myeloid leukemia (AML) patients exposed to 1 mM SB, 75-, 100- and 150 nM FP or their corresponding combinations during 24 h. Apoptotic cells were identified by morphological analysis of Wright and Giemsa-stained cytospin preparations as described in the text.

FIGS. 11A and B. FP/SAHA co-treatment works as a potent cell death inducer combination. A, percent death of cells observed in U937 myelomonocytic human leukemia cells exposed to 1 μM SAHA, 100 or 150 nM FP or the combination of both compounds from 0.5 to 24 h. B, percentage of FP/SAHA-induced cell death in HL-60 promyelocytic cells. C=control; S1=1.0 μM SAHA; S1.5=1.5 μM SAHA.

FIGS. 12A and B. A, the loss of mitochrondrial membrane potential (Δψₘ) was monitored at different time points in cells exposed to 100 nM FP, 1 μM SAHA or the combination of both. Values represent the means for three separate experiments±SE an are expressed as the percentage of cells expressing low mitochondrial membrane potential (Δψₘ) as manifested by reduced levels of DioC₆ uptake relative to untreated controls. B, cell cycle analysis of cells after exposure to FP/SAHA alone or in combination. U937 cells were incubated in 70% ethanol and then treated with propidium iodide and subjected to cell cycle analysis using a Becton Dickinson FACScan flow cytometer and ModFit software, as described in MM. The results of a representative study are shown; additional experiments yielded similar results.

FIG. 13. Flavopiridol in combination with four different histone deacetylase inhibitors works as a potent inducer of cell death. Percent death of cells observed in U937 myelomonocytic human leukemia cells exposed to 100 nM FP alone or in combination with 1 mM SB, 1 μM SAHA, 50 nM tricostatin A (TSA) and 20 nM depsipeptide (Depsi) after 24 h of treatment.

FIGS. 14A and B. FP/all trans retinoic acid (ATRA) co-treatment works as a potent apoptosis inducer and effects the loss of mitochrondrial membrane potential. A, percent apoptosis observed in HL60 human leukemia cells exposed to 80 μM nM FP and 0, 3, 5, or 10 μM ATRA. B, loss of mitochrondrial membrane potential in HL60 human leukemia cells exposed to 80 μM nM FP and 0, 3, 5, or 10 μM ATRA. Values represent the means for three separate experiments±SE an are expressed as the percentage of cells expressing low mitochondrial membrane potential (Δψₘ) as manifested by reduced levels of DioC₆ uptake relative to untreated controls.

### DETAILED DESCRIPTION OF THE PREFERRED EMBODIMENTS OF THE INVENTION

This invention is the result of the unexpected discovery that, contrary to expectations, cyclin dependent kinase inhibitors oppose, rather than promote, differentiation inducing agent induced maturation events. In fact, co-administration to cancer cells of a cyclin dependent kinase inhibitor and an agent that induces cellular differentiation results in the promotion of apoptosis of the cancer cells to an extent significantly greater than that which is observed with a cyclin dependent kinase inhibitor alone. Furthermore, the co-administration results in dysregulation of various proteins and signaling pathways associated cell cycle arrest and differentiation.

The present invention thus provides a method for promoting apoptosis in cancer cells by the co-administration of a cyclin dependent kinase inhibitor and an agent that induces cell differentiation or maturation. The present invention further provides a method of treating cancer in a patient by co-administering a cyclin dependent kinase inhibitor and an agent that induces cell differentiation.

By “cyclin dependent kinase inhibitor” we mean that the primary activity of the compound, as recognized by those of skill in the art, is to inhibit cyclin dependent kinases. Those of skill in the art will recognize that many types of cyclin dependent kinase inhibitor exist which can be utilized in the practice of the present invention. Examples of such inhibitors include but are not limited to flavopiridol, UCN-01, roscovitine, olomoucine, and butyrolactone, and the like. Any CDK inhibitor may be utilized in the practice of the present invention, so long as the inhibitor exhibits the property of inducing apoptosis in cancer cells when co-administered with an agent that induces cellular differentiation.

Likewise, those of skill in the art will recognize that many types of agents that induce cellular differentiation exist which can be utilized in the practice of the present invention. By “agents that induce cellular differentiation” we mean that the primary activity of the compound, as recognized by those of skill in the art, is to induce cellular differentiation. By “cellular differentiation” we mean differentiation that entails an irreversible commitment of the cell to cell cycle arrest, acquisition of mature features, and the loss of self-renewal capacity. Examples of such agents include but are not limited to including histone deacetylase inhibitors [such as sodium butyrate, phenylbutyrate, suberoylanilide hydroxamic acid (SAHA), depsipeptide, tricostatin A, MS-275, and CI-994], protein kinase C activators [such as PMA and bryostatin], retinoids [such as all trans retinoic acid (ATRA)]), and Vitamin D3. Any agent which induces cellular differentiation may be utilized in the practice of the present invention, so long as the agent exhibits the property of inducing apoptosis in cancer cells when co-administered with A cyclin dependent kinase inhibitor.

By “co-administration” or “co-administering” we mean that the two agents are administered in temporal juxtaposition. The co-administration may be effected by the two agents being mixed into a single formulation, or by the two agents being administered separately but simultaneously, or separately and within a short time of each other. For example, in general the two agents are co-administered within the time range of 24-72 hours. In this case, the agents may be administered in either order, i.e. the agent that induces cellular differentiation may be administered first, or the cyclin dependent kinase inhibitor may be administered first. In a preferred embodiment of the instant invention, the two agents are co-administered in a single formulation, or are co-administered simultaneously. Further, more than one cyclin dependent kinase inhibitor or more than one agent that induces cellular differentiation may be administered.

By “promoting apoptosis” we mean that the level of apoptosis occurring in the targeted cancer cells upon simultaneous exposure of the cancer cells to a cyclin dependent kinase inhibitor and an agent that induces cell differentiation is greater than the level of apoptosis that would occur in the presence of either agent alone. In general, the increase in the level of apoptosis will be in the range of about 10% to 100%. In a preferred embodiment of the present invention, the increase in the level of apoptosis will be in the range of about 40% to 80%. In yet another preferred embodiment, the increase is in the range of about 50% to 70%. Those of skill in the art will recognize that it is possible to quantitate the level of apoptosis in cancer cells by several means which are well-known and readily available, including morphological assessment of Wright and Giemsa-stained cytospin preparations, and TUNEL assays. The effects may be assessed in vivo or in vitro.

The methods described herein can be used for promoting apoptosis in and treating cancers of a number of types, including but not limited to breast cancer, sarcomas and other neoplasms, bladder cancer, colon cancer, lung cancer, various leukemias and lymphomas, multiple myeloma, etc.

One skilled in the art will recognize that the amount of a cyclin dependent kinase inhibitor and an agent that induces cell differentiation to be co-administered will be that amount sufficient to induce apoptosis in the targeted cancer cells. Such an amount may vary inter alia depending on such factors as the gender, age, weight, overall physical condition, of the patient, etc. and must be determined on a case by case basis. The amount may also vary according to the type of cancer being treated, and the other components of the treatment protocol (e.g. other forms of chemotherapy, surgery, and the like. Generally, a suitable dose is one that results in a concentration of the active agents at the site of a tumor in the range of about 50 nM-10 μM, and more usually from 100 nM to 5 μM. It is expected that serum concentrations of about 1 μM should be sufficient in most cases. Those of skill in the art will recognize that such details are normally worked out during clinical trials.

Co-administration of the agents may be oral, perenteral or topical. The term parenteral as used herein includes subcutaneous injections, intravenous, intramuscular, intraarterial injection, or infusion techniques. The agents may be administered in any of several forms, including tablets, pills, powders, lozenges, sachets, cachets, elixirs, suspensions, emulsions, solutions, syrups, aerosol (as a solid or in a liquid medium), soft or hard gelatin capsules, suppositories, sterile injectable solutions and sterile packaged powders.

All pharmaceutical compositions of the agents utilized in the practice of the present invention may also include a pharmaceutically acceptable carrier. The agents may be mixed with a carrier, or diluted by a carrier, or enclosed within a carrier which may be in the form of a capsule, sachet, paper or other container. When the carrier is a diluent, it may be a solid, semisolid or liquid material which acts as a vehicle, excipient or medium for the inhibitor. Some examples of suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, celluose, water syrup, methyl cellulose, methyl and propylhydroxybenzoates, talc, magnesium stearate and mineral oil. The formulations can also include lubricating agents, wetting agents, emulsifying agents, preservatives, and sweetening or flavoring agents.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol.

In another aspect, the present invention contemplates a pharmaceutical composition comprising a cyclin dependent kinase inhibitor and an agent that induces cellular differentiation. Examples of appropriate carriers include those which are listed above. Examples of cyclin dependent kinase inhibitors include but are not limited to flavopiridol, UCN-01, roscovitine, olomoucine, and butyrolactone etc. Examples of agents that induces cellular differentiation include but are not limited to histone deacetylase inhibitors [such as sodium butyrate, phenylbutyrate, suberoylanilide hydroxamic acid (SAHA), depsipeptide, tricostatin A, MS-275, and CI-994], protein kinase C activators [such as PMA and bryostatin], retinoids [such as all trans retinoic acid (ATRA)], and Vitamin D3. Such a composition also comprises a carrier suitable for in vivo administration, examples of which are listed above.

The following examples provide illustrations of the practice of the present invention but should not be construed so as to limit the invention in any way.

### EXAMPLES

### Materials and Methods

Drugs, Biologicals and Chemical Reagents. PMA (Sigma; St. Louis, Mo.) was dissolved in dimethyl sulfoxide (DMSO) and aliquots stored at −20° C. FP (L86 8275; NCS 649890) is readily available from the National Cancer Institute or CTEP. FP was formulated in DMSO and 10⁻² M stock solutions stored at −20° C. Sodium butyrate was supplied as a powder (Calbiochem, La Jolla, Calif.) and dissolved in PBS prior to use. Suberoylanilide hydroxamic acid (SAHA) was dissolved in DMSO. The mitochondrial dye 3,3-dihexlyoxacarbocyanine (DiOC₆) was purchased from Molecular Probes (Eugene, Oreg.). The antimetabolite 1-β-D-arabinofuranosylcytosine (ara-C) was dissolved in sterile PBS and 10⁻³ M aliquots stored at 4° C. Hygromycin B was obtained from Boehringer Mannheim (Mannheim, GR). PD 98059 and U0126 were purchased from Calbiochem (La Jolla, Calif.), and formulated in DMSO according to the manufacturer's instructions. The pancaspase inhibitor BOC-Asp(OMe)-Fluoromethyl Ketone (B-D-FMK) was purchased from Enzyme Systems Products (Livermore, Calif.). Primary antibody for phosphorylated extracellular-regulated kinases 1 and 2 (ERK1 and 2) was provided in the PhosphoPlus® p44/p42 MAP Kinase Antibody Kit (New England Biolabs; Beverly, Mass.). Primary antibodies for p21, p27, and actin were purchased from Transduction Laboratories (Lexington, Ky.). Primary antibodies for underphosphorylated pRB and procaspase 3 were obtained from Pharmingen (San Diego, Calif.). The primary antibody for poly(ADP-ribose)polymerase (PARP) was purchased from Biomol Research Laboratories (Plymouth Meeting, Pa.). Secondary antibodies conjugated to horseradish peroxidase were obtained from Kirkegaard and Perry Laboratories, Inc. (Gaithersburg, Md.). Coomassie protein assay reagent was purchased from Pierce (Rockford, Ill.) and an enhanced chemiluminescence kit was obtained from New England Nuclear (Boston, Mass.). Hypoosmolar buffer for electroporation was purchased from Eppendorf Scientific, Inc. (Westbury, N.Y.) and luciferase assay reagents were obtained from Promega (Madison, Wis.). All other chemicals or reagents were purchased from Sigma Chemical Company (St. Louis, Mo.).

Cell culture. The myelomonocytic leukemia cell line U937 was derived from a patient with histiocytic lymphoma and was obtained from American Type Culture Collection. The HL-60 cell line was derived from a patient with acute promyelocytic leukemia as previously described [Grant et al. 1992]. U937 and HL-60 cells were maintained in logarithmic growth phase by culturing in suspension in phenol red-free RPMI-1640 (Life Technologies, Inc., Grand Island, N.Y.) and 10% (v/v) fetal calf serum (Hyclone, Logan, Utah), in a humidified atmosphere (95% air/5% CO₂) at 37° C. To obtain antisense-expressing cell lines, U937 cells were transfected by electroporation with a pREP4 vector (Invitrogen; Carlsbad, Calif.) or the pREP4 vector containing the p21^{WAF1/CIP1} coding region in an antisense orientation as previously described [Wang et al. 1999]. Transfectant U937 leukemia cells stably overexpressing the anti-apoptotic protein Bcl-2 were obtained as previously reported [Wang et al. 1997]. These cells, designated as U937/Bcl-2, were generated along with their empty-vector counterparts (i.e., U937/pCEP4). U937 transfectant cell lines were maintained as described above in the presence of hygromycin B (400 μg/ml) and transferred to selection-free media 24 h prior to experimentation. All experiments were performed on cells in logarithmic phase.

Cell Cycle Analysis. Following drug treatment, cells were pelleted by centrifugation at 500 g×6 min, and resuspended in 70% ethanol. The cells were incubated on ice for at least 1 h and resuspended in 1 ml cell cycle buffer (0.38 mM Na-Citrate, 0.5 mg/ml RNase A, and 0.01 mg/ml propidium iodide) at a concentration of 10×10⁵ cells/ml, stored in the dark at 4 C until analysis (24 h), using a Becton-Dickinson FACScan flow cytometer and Verity Winlist software (Verity Software, Topsham, Me.).

Morphological assessment of apoptosis. U937 cells were evaluated for apoptosis by morphological assessment of Wright and Giemsa-stained cytospin preparations. At designated times, cells were transferred to slides by cytocentrifugation, fixed, stained, and evaluated under light microscopy for treatment-induced apoptosis. Apoptotic cells were identified by classical morphologic features (i.e., nuclear condensation, cell shrinkage, and formation of apoptotic bodies). Five or more randomly selected fields, encompassing a total≧500 cells/slide, were evaluated to determine the percentage of apoptotic cells for each treatment condition. The extent of apoptosis determined by this method has been shown to correlate closely with results obtained using the TUNEL assay (unpublished observations).

Assessment of drug interactions. Interactions between PMA and FP were characterized as previously described by Chou and Talalay [1984]. The percentages of apoptotic cells were assessed following PMA (1-10 nM) or FP (10-100 nM) treatment alone or in combination for 24 h at a constant PMA/FP dose ratio (1/10). The combination index (C.I.) was determined using Median Dose Effect Analysis Software (Elsevier Biosoft; Cambridge, UK). C.I. values<1 correspond to synergistic drug interactions.

Western analysis. Equal quantities of protein (15 μg/condition for MAPK, 40 μg/condition for cytochrome c, or 25 μg/condition) were separated by SDS-PAGE [underphosporylated pRB (7.5%), poly(ADP-ribose)polymerase or PARP (8.0%), p44/p42 MAPK (10%), p21, p27, procaspase 3 (12%), and cytochrome c (15%)] and electroblotted onto nitrocellulose. Western analysis of p44/p42 MAPK protein was performed according to the manufacturer's instructions (New England Biolabs); otherwise, studies were performed as previously described [Wang et al, 1998]. Briefly, U937 cells (5×10⁶) were pelleted by centrifugation, resuspended in 50 μl PBS, lysed by the addition of 2× Laemmli buffer and boiled for 5 min. Proteins were quantified with Coomassie protein assay reagent. Blots were blocked in PBS-T/5% milk, washed twice with PBS-T, and incubated overnight at 4° C. with the appropriate primary antibody. The blots were washed with PBS-T and incubated with a horseradish-peroxidase conjugated secondary antibody diluted appropriately in PBS-T/5% mill. Following incubation, blots were developed by enhanced chemiluminescence exposure to Kodak X-OMAT film (Eastman Kodak Co., Rochester, N.Y.) and reprobed with antibodies directed against actin to control for equal loading of protein.

Alternatively, whole cell-pellets were washed twice in PBS, resuspended in PBS, and lysed by the addition of I volume of loading buffer. Lysates were boiled for 10 min, centrifuged at 12,800 g for 5 min and quantified using Coomassie protein assay reagent (Pierce, Rockfold, Ill.). 25 μg of total proteins per point were separated by SDS-PAGE and electro-blotted to nitrocellulose. The blots were blocked in 5% non-fat milk in PBS-T and probed for 1 h with the appropriate dilution of primary antibody. Blots were washed 3×10 min in PBS-T and then incubated with a 1:2000 dilution of peroxidase-conjugated secondary antibody for 1 h at room temperature. Blots were again washed 3×10 min in PBS-T and then developed by enhanced chemiluminesence. Where indicated, blots were stripped and re-probed with antibodies directed against actin.

Primary antibodies for both p21^{WAF1/CIP1} and p27^{KIP1} (Transduction Laboratories, Lexington, Ky.) were used in a 1:1000 dilution; PARP (1:1000; Oncogene Research Products, Cambridge, Mass.); pro-caspase 3 (1:1000; Transduction Laboratories); cytochrome c (1:2000; Pharmingen); bcl-2 (1:2000; DAKO, Denmark); bax (1:1000; Santa Cruz Biotechnology, Inc., Santa Cruz, Calif.); bid (1:1000; Cell Signaling, Beverly, Mass.); XIAP (1:1000; Cell Signaling); Mcl-1 (1:1000; Pharmingen); bak (1:1000; Calbiochem); c-Myc (1:1000; Pharmingen, San Diego, Calif.); actin (1:2000; Sigma Chemicals, St. Louis, Mo.); cyclins-D1, -A and -E (1:1000; Pharmingen); pRb, underP-pRb, pRb-CDK2 and pRb-CDK4 (all 1:1000; Pharmingen); Ac-H3 and Ac-H4 (both 1:1000; Upstate Biotechnology, Lake Placid, N.Y.); E2F-1 (Upstate Biotechnology). Secondary antibodies conjugated to horseradish peroxidase were obtained from Kirkegaard and Perry Laboratories, Inc. (Gaithersburg, Md.). Coomassie protein assay reagent was purchased from Pierce (Rockford, Ill.), and an enhanced chemiluminescence kit was obtained from New England Nuclear (Boston, Mass.). The pan-caspase inhibitor BOC-D-fmk and the caspase 8 inhibitor Z-IETD-fmk were purchased from Enzyme System Products (Livermore, Calif.) All other chemicals or reagents were purchased from Sigma.

Assessment of mitochondrial membrane potential. At designated times, 1 ml aliquots of cells (2×10⁵) were harvested and incubated with 40 nM DiOC₆ for 15 minutes at 25° C. as previously described [Zamzami et al 1995]or at 37° C. Samples were analyzed using a Becton Dickinson FACScan flow cytometer (excitation λ=488 nm; emission λ=525 nm). Results were expressed as the percentage of total cells exhibiting loss of mitochondrial membrane potential (ΔΨₘ), manifested by a reduction in DiOC₆ uptake relative to untreated controls. Data acquisition and analysis was performed using CELLQUEST Software (Becton Dickinson, Mansfield, Mass.).

Cytochrome c release. The assay for cytochrome c release was performed as previously described by Single et al. [1998] with modifications. At designated times following drug treatment, 4×10⁶ cells were washed with PBS and resuspended in 50 μl of assay buffer (75 mM NaCl, 1 mM NaH₂P0⁴, 8 mM Na₂HP0⁴, 1 mM EDTA, 250 mM sucrose) containing digitonin (700 μg/ml). Three minutes following addition of digitonin assay buffer, cells were pelleted by centrifugation. The supernatants were transferred to tubes containing 2× Laemmli buffer (50 μl) and boiled for 5 minutes. Proteins were separated by SDS-PAGE as described above.

Clonogenic survival assays. Clonogenic survival assays assessed the effects of 10 nM PMA, FP (50 or 100 nM) or PMA/FP co-treatments on leukemic cell self-renewal capacity. Cell densities were determined after 24-h drug treatment using a Coulter Counter (Coulter Electronics, Hialeah, Fla.). Cells were pelleted by centrifugation and resuspended in fresh media to achieve a final cell density of 3×10⁵/ml. A total of 4500 cells were plated in soft agar cloning medium for each condition as previously described [Powell et al. 1995]. Colonies, defined as groups of≧50 cells, were scored after 12 days of incubation. Clonogenic survival in drug-treated samples was expressed as a percentage relative to untreated controls.

Differentiation studies. Expression of the monocytic differentiation marker, CD11b, was monitored by direct immunofluorescence staining and flow cytometric analysis as previously described [Freemerman et al. 1997]. Following drug treatment, suspension and adherent cells were enumerated by a Coulter Counter and 2×10⁶ cells were pelleted by centrifugation. The supernatant was aspirated and the cells were resuspended in 300 μl of ice-cold PBS. Two 100 μl aliquots from each sample were then combined with either phycoerythrin-1 (10 μl) or the IgG control. Samples were incubated for 20 min at 4° C. and diluted in PBS (1 ml). Sample data were collected using a Becton Dickinson FACScan flow cytometer and analyzed with Verity Winlist Software (Verity; Topsham, Me.). Differentiation was also monitored by determining the percentage of U937 cells exhibiting plastic adherence following drug treatments as previously described [Vrana et al. 1998]. p21^{WAF1/CIP1} Luciferase Reporter Assay. U937 cells were transfected with a full-length p21^{WAF1/CIP1} promoter (base pairs 1-2326) as described by Chinery et al. [Chinery et al. 1997] utilizing electroporation (Eppendorf Multiporator, 620V, 60 μs) in hypoosmolar buffer. In each experiment, pSV-βgal (Promega), containing the β-galactosidase gene under the control of the constitutively active SV40 promoter and enhancer, was co-transfected with the luciferase reporter at a ratio of 1:5 (w/w) to normalize for transfection efficiency. The cells were incubated overnight in complete media and treated with 10 nM PMA, 100 nM FP, or 10 nM PMA/100 nM FP for 3 or 6 h. At indicated times, cells were washed in serum-free media, resuspended in 0.25M Tris-HCl pH 7.8, lysed by rapid freeze-thaw, centrifuged and the supernatant frozen at −70° C. Lysates (20 μL) were mixed with luciferase assay reagent (100 μL) and monitored for 20 seconds in a luminometer (Monolight 2010). For β-galactosidase activity, lysates (50 μL) were mixed in triplicate with an equal volume of luciferase assay buffer (2×) in a 96-well plate and incubated overnight. The reaction was terminated by addition of NaCO₃ and absorbance was measured at 405 nm in a Vmax plate reader (Molecular Devices). Activity was determined by comparison to a standard curve. Luciferase reporter activity in RLU was normalized to mU of β-galactosidase activity as described by Rosenthal [Rosenthal 1987].

Electrophoretic Mobility Shift Assay (EMSA) and Immunoprecipitation (IP). Whole-cell extracts were prepared as previously described [Bagchi et al. 1990] and EMSA following IP was performed as previously reported [Chellapan et al. 1991]. Whole cell extracts (150 μg) were combined with 4 μl of anti-RB monoclonal antibody (Calbiochem) for the IP. Immunoprecipitated proteins were recovered on Protein A sepharose beads and dissociated by treatment with deoxycholate. The presence of E2F in the IP was monitored by EMSA as previously noted [Yee et al. 1987].

Statistical analysis. Significant differences between experimental values were determined using the student's T test.

Histone Deacetylase (HDAC) Assay. Acid extraction of proteins from treated cells and detection of acetylated histones H3 and H4 by Western blot analysis was performed per the manufacturer's instructions (Upstate Biotechnology). 10 μg of extract was loaded per lane and separated on pre-cast 4-20% Biorad Gradient Gels.

Analysis of cytosolic cytochrome c. A previously described technique was employed.³² After treatment, cells (5×10⁷/condition) were harvested by centrifugation at 600 g for 10 min at 4° C. The S-100, or cytosolic fraction, was prepared as described with minor modifications. Cell pellets were washed once with ice-cold phosphate-buffered saline and resuspended in five volumes of buffer A (20 mM Hepes-KOH, pH 7.5, 10 mM KCl, 1.5 mM MgCl2, 1 mM sodium EDTA, 1 mM sodium EGTA, 1 mM dithiothreitol, 0.1 mM phenylmethlysufonyl fluoride, and 250 mM sucrose; all Sigma. After chilling for 30 min on ice, the cells were disrupted by 15 strokes of a Potter-Elvehjem (PTFE-pestle) homogenizer. The homogenate was then centrifuged twice to remove unbroken cells and nuclei (3000 g for 10 min at 4° C.). The resulting supernatant was further centrifuged at 100,000 g for 1 h at 4° C., and the supernatants, designated as S-100 fractions, were immediately subjected to Western analysis as described above. For each condition, 25 μg of the S-100 fraction was loaded on the gel, and probed with the corresponding antibody.

Immunoprecipitation and EMSA. Whole-cell extracts were prepared by hypotonic shock followed by salt extraction, as described previously (Chellappan et al., 1991). Portions (50 to 200 μg) of whole-cell extracts were treated with 5 μl of the appropriate primary antibody in a volume of 100 μl at 4° C. for 1 h. Then, 3 mg of protein A-Sepharose or protein G-Sepharose in a 100-μl volume was added and incubated for an additional hour. The binding was performed in a buffer containing 20 mM HEPES (pH 7.9), 40 mM KCl, 1 mM MgCl2, 0.1 mM EGTA, 0.1 mM EDTA, 0.1 mM DTT, 0.1 mM NaF, 0.1 mM Na3VO4, 0.5% NP-40, and 3 mg of BSA per ml. The beads were washed six times with 600 μl of the same buffer and boiled in 20 μl of SDS sample buffer. Electrophoretic mobility shift assay (EMSA) after immunoprecipitation was performed as previously described (Chellappan et al., 1991, Zhang & Chellappan, 1995). An EcoRI-HindIII fragment of the adenovirus E2 promoter containing two E2F binding sites (TTTCGCGC) was end labeled by using Klenow fragment and was used as the probe in all of the assays. Briefly, 8 μg of whole-cell extracts were incubated with approximately 0.2 ng of 32P-labeled E2F probe in a buffer containing 20 mM HEPES (pH 7.9); 40 mM KCl; 0.1 mM concentrations each of MgCl2, EGTA, EDTA, DTT, NaF, and Na3VO4; 1% NP-40, 1 μg of salmon sperm DNA per ml, and 10 μg of BSA per ml. After incubation at room temperature for 20 min, the reactions were separated on a 4% polyacrylainide gel in 0.25×TBE at 300 V for 3 h. The gel was dried, and the bands were detected by autoradiography.

Isolation of Mononuclear Cells. Equivolume amounts of peripheral blood and 37E cell culture media (RPMI 1640) were mixed in a centrifuge tube. An equal volume of 37E sterile ficoll (Histopaque-1077, Sigma-Aldrich, St. Louis, Mo.) was added and the tubes were spun for 30 minutes at 400×g at room temperature. Cells were then harvested from the interface between the serum and the ficoll, washed in media, and counted.

### EXAMPLE 1

### FP and PMA Promote Apoptosis in Human Leukemia Cells

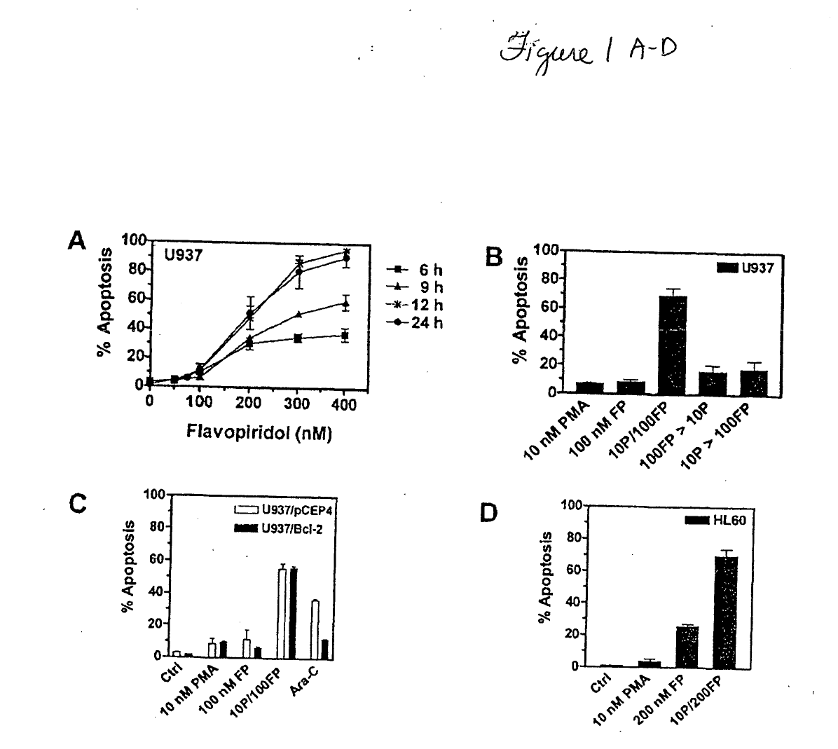

To evaluate the dose response for FP-induced apoptosis, U937 cells were exposed to various concentrations of FP (e.g., 50-400 nM) for 6, 9, 12, or 24 h, after which apoptosis was determined by morphologic assessment of Wright and Giemsa-stained cytospin preparations (FIG. 1A). U937 cells were susceptible to apoptosis following chronic exposures (e.g., 12 or 24 h) to FP concentrations≧200 nM, whereas concentrations≦100 nM minimally induced apoptosis (e.g., ≦10% of cells), regardless of the treatment interval. Subsequently, the extent of apoptosis was assessed in U937 cells exposed to 100 nM FP in combination with a minimally toxic concentration of PMA (10 nM). Three schedules were evaluated: (a) co-administration of the agents for 24 h; (b) 10 nM PMA for 24 h, followed by 100 nM FP for 24 h; or (c) 100 nM FP for 24 h, followed by 10 nM PMA for 24 h.

Co-administration of 10 nM PMA and 100 nM FP for 24 h (FIG. 1B) resulted in a significant increase in apoptosis (69%; p<0.0001); in contrast, sequential schedules only marginally increased cell death (apoptosis<20%). Co-treatment with PMA/FP also effectively induced apoptosis in U937 cells overexpressing the anti-apoptotic protein Bcl-2 (56%) whereas these cells were significantly protected from the lethal effects of the antimetabolite ara-C (1-β-D-arabinofuranosylcytosine; 1.0 μM×6 h; FIG. 1C). Co-administration of FP (200 nM) and PMA (10 nM) also resulted in a substantial increase in apoptosis (70%; p<0.0004) in the human promyelocytic leukemia cell line HL-60 (FIG. 1D).

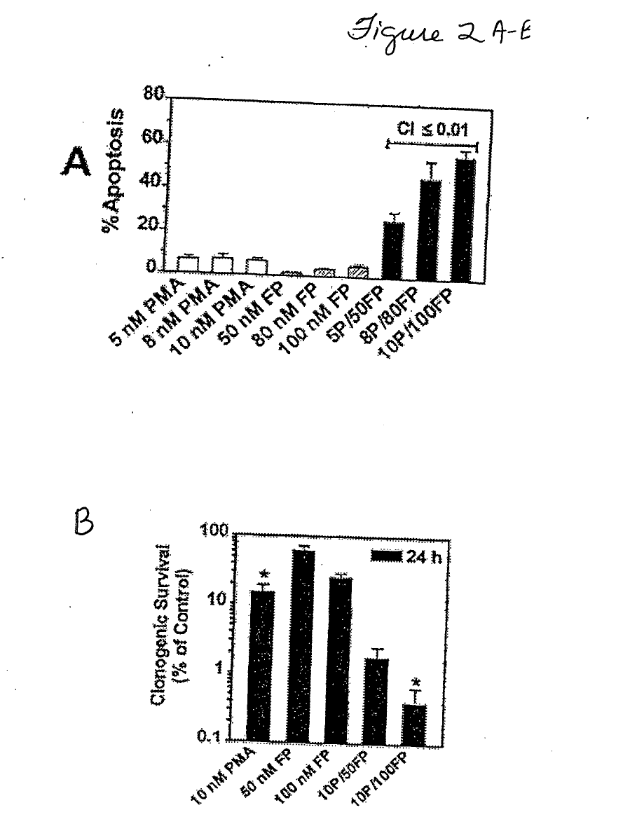

Median dose effect analysis [Chou and Talahay, 1984] was used to characterize interactions between FP and PMA over a range of drug concentrations with respect to induction of apoptosis (FIG. 2A). C.I. values were consistently<0.01, indicating a highly synergistic drug interaction. Evidence for enhanced proteolytic cleavage of procaspase-3 [Li et al. 1997] and degradation of PARP, one of the major caspase-3 substrates [Tewari et al. 1995], was apparent at each time interval examined for cells exposed to PMA/FP (data not shown). Lastly, the increase in apoptosis following PMA/FP co-treatment was accompanied by a marked reduction in clonogenic survival (FIG. 2B). The greatest reduction in clonogenic survival occurred with the 10 nM PMA/100 nM FP combination, which was significantly more inhibitory than the effects of PMA alone (<1% versus 15%; p<0.03).

Together, these findings demonstrate that co-administration of minimally toxic concentrations of a histone acetylase inhibitor and an agent that promotes cancer cell defferentation unexpectedly
1) provides a potent apoptotic stimulus in several human myeloid leukemia cancer cell types (including those expressing high levels of Bcl-2);
2) promotes caspase activation in human myeloid leukemia cancer cells; and
3) causes a marked decline in leukemic cancer cell clonogenicity.

### EXAMPLE 2

### FP Combined with PMA Promotes Mitochondrial Damage Upstream of Caspase Activation

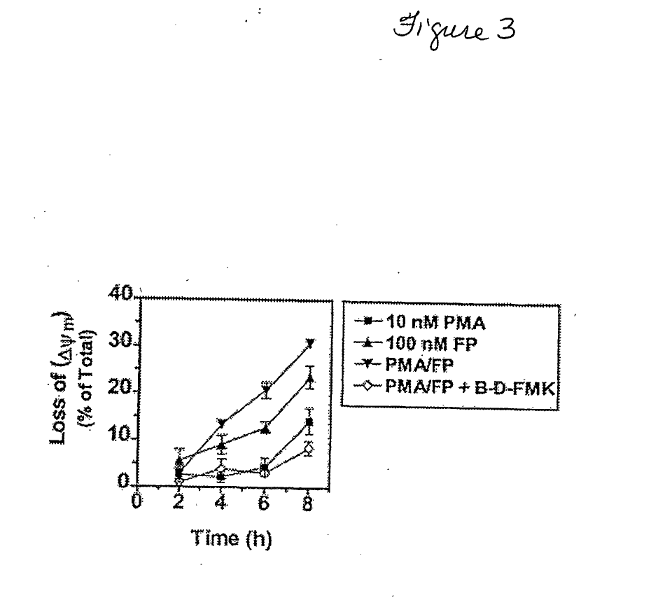

The effects of FP and PMA on mitochondrial injury [e.g., loss of mitochondrial membrane potential (Δψₘ)], an event that can precede the morphological features of apoptosis [Marchetti et al. 1996], was assessed at early time points (e.g., 2-8 h; FIG. 3A). The combination of FP (100 nM) and PMA (10 nM) triggered the greatest decline in Δψₘ at the 4-8 h exposure intervals, although reductions were noted in cells treated with FP alone at these times. Co-administration of the pancaspase inhibitor, BOC-Asp(OMe)-fluoromethyl ketone (B-D-FMK; 20 μM), effectively blocked the loss of Δψₘ in cells exposed to PMA and FP, suggesting that this event represents a consequence of caspase activation. In addition, U937 cells treated with the combination of PMA and FP for 6 h displayed a more pronounced release of cytochrome c into the cytosol than cells treated with PMA or FP alone, although FP alone had some effect (data not shown). However, in contrast to the loss of Δψₘ, the cytochrome c release induced by PMA/FP co-treatment was not blocked by B-D-FMK (20 μM).

Together, these findings demonstrate that co-administration of FP (100 nM) with PMA (10 nM) promotes early mitochondrial damage in human leukemia cells and suggest that release of cytochrome c into the cytoplasm represents an upstream event in the apoptotic process.

### EXAMPLE 3

### FP Antagonizes PMA-Induced Differentiation

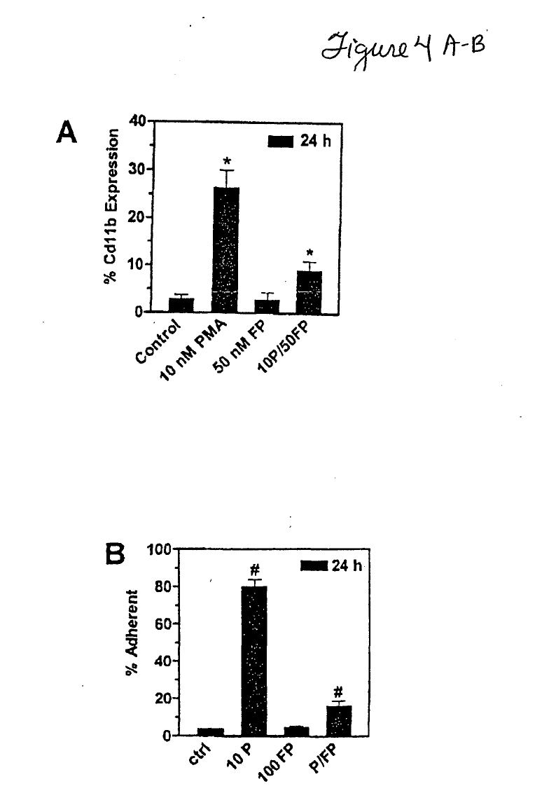

Induction of leukemic cell maturation can be accompanied by apoptosis [Gunji et al. 1992]; therefore, it is conceivable that PMA/FP-mediated cell death could result from enhanced cellular differentiation. Thus, the effects of FP were examined with respect to PMA-related induction of the monocytic maturation marker, CD11b (FIG. 4A). FP did not enhance PMA-mediated induction of CD11l at 24 h; instead, FP co-treatment significantly reduced the percentage of cells displaying CD11b expression from 26% to 9.0% (p<0.03). FP (100 nM) co-treatment also significantly decreased PMA-induced plastic adherence, another manifestation of U937 cellular maturation, from 80% to 16% (p<0.001), (FIG. 4B).

Thus, despite its CDK inhibitory activity, FP significantly antagonized PMA-induced differentiation in U937 cells.

### EXAMPLE 4

### Effects of FP on pRb and CDKI Expression in PMA-Treated Cells

The effects of FP on proteins involved in the PMA-induced G₁ arrest program (e.g., pRb and the CDKIs p21^{WAF1/CIP1} and p27^{KIP1}) were examined by Western analysis. Exposure of cells to 10 nM PMA for 24 h resulted in a significant increase in the underphosphorylated pRb species, whereas FP alone (50, 80 or 100 nM) had a minimal effect on pRb dephosphorylation at 24 h. When cells were co-exposed to PMA (10 nM) and FP (50, 80 or 100 nM), there was a marked reduction in full-length underphosphorylated pRb (≈Mᵣ 110,000), which was accompanied by the appearance of an immunoreactive Mᵣ 68,000 band. The latter presumably corresponds to a pRb cleavage product resulting from the actions of interleukin 1β-converting enzyme-like proteases in leukemic cells undergoing apoptosis [An and Dou, 1996]. Similar findings were obtained when total pRb was examined following co-treatment with PMA and FP for 24 h (data not shown). Furthermore, exposure of U937 cells to 10 nM PMA for 24 h resulted in a significant increase in p21^{WAF1/CIP1} expression and a slight increase in p27^{KIP1} expression as shown by Western analysis while FP alone had no effect on the expression of these CDKIs. In contrast, marked reductions in levels of p21^{WAF1/CIP1} and p27^{KIP1} were observed after 24 h of co-treatment, particularly at the highest FP concentration examined (e.g., 100 nM).

Thus, 24-h exposure to FP significantly reduced the amount of full-length underphosphorylated pRb induced by PMA, and markedly diminished expression of the CDKIs p21^{WAF1/CIP1} and p27^{KIP1}.

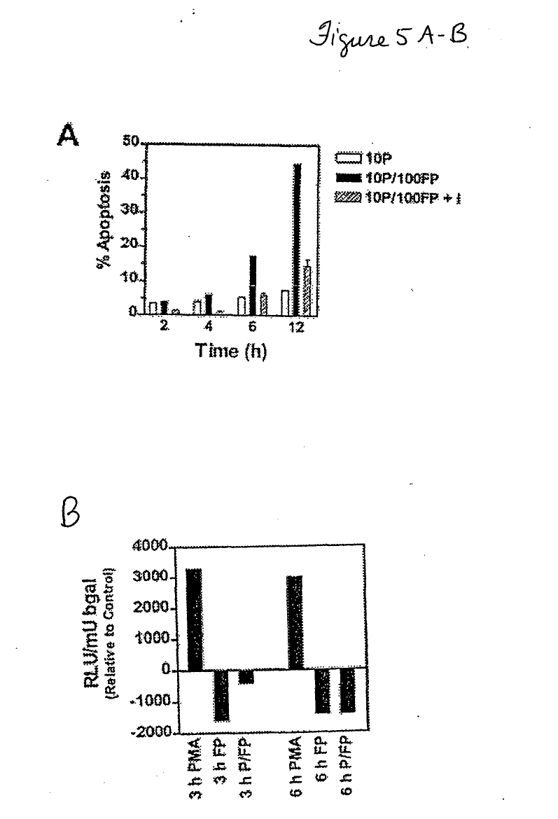

Additional studies were conducted at early time intervals to determine if observed reductions in CDKI expression following PMA/FP co-treatment were the result of caspase activation during apoptosis, a phenomenon that has previously been described [Zhang et al, 1999]. U937 cells were exposed to the combination of 10 nM PMA and 100 nM FP in the presence or absence of the pancaspase inhibitor B-D-FMK (20 μM), and the extent of apoptosis (FIG. 5A) or CDKI protein expression (data not shown) was assessed over the ensuing 2-12 hr. Addition of 20 μM B-D-FMK effectively antagonized apoptosis in cells exposed to PMA and FP for the duration of the time course. The apoptosis percentage after 12 h of co-treatment was 15% and 45% in the presence or absence of 20 μM B-D-FMK, respectively (FIG. 5A). As shown by Western analysis, FP significantly attenuated PMA-induced p21^{WAF1/CIP1} expression in the presence or absence of 20 FM B-D-FMK, indicating that FP interferes with p21^{WAF1/CIP1} through a caspase-independent mechanism. In contrast, p27^{KIP1} was cleaved by caspases within 4 h of PMA/FP co-treatment, based upon the observation that formation of a recently described [Loubat et al. 1999] 23 kDa cleavage fragment was prevented by the addition of 20 μM B-D-FMK. Finally, transient transfection of U937 cells with a p21^{WAF1/CIP1} luciferase reporter plasmid containing the full-length promoter revealed that PMA alone markedly increased p21^{WAF1/CIP1} reporter activity at 3 and 6 h, whereas co-incubation of FP reduced activity to below basal levels (FIG. 5B).

This finding suggests that FP-mediated antagonism of p21^{WAF1/CIP1} expression may occur at the transcriptional level.

### EXAMPLE 5

### Early Effects of FP on p21^{WAF1/CIP1}, pRb, and Phospho-ERK in PMA-Treated Cells

Parallel time course studies were conducted to examine early FP-mediated perturbations in the expression of various differentiation-related proteins, including p21^{WAF1/CIP1}, underphosphorylated pRb, and phosphorylated p42/p44 MAPK (i.e., ERK1 and ERK2). Consistent with previous observations, PMA (10 nM) induced p21^{WAF1/CIP1} in U937 cells within 1 h of drug addition, and expression was very marked by 3-6 h; moreover, induction of this CDKI was substantially attenuated by FP (100 nM) co-administration. In addition, FP co-treatment accelerated the temporal pattern of pRb dephosphorylation relative to PMA alone by approximately 2 h (e.g., 30 min versus 3 h), consistent with the ability of FP to inhibit multiple CDKs [Senderowicz, 1999]. Evidence of pRb cleavage to a 68 kDa subfragment, as well as PARP degradation (data not shown), was discernible at 6 h in cells treated with the combination of PMA and FP. Early alterations in MAPK phosphorylation (activation) were also observed. Thus, treatment of cells with PMA alone resulted in an increase in phosphorylated ERK1/2 that was apparent after 30 min, maximal after 1 h, and which declined slightly over the ensuing 5 h. Treatment with FP alone modestly increased ERK activation at 3-6 h. However, co-administration of FP with PMA led to a more pronounced and sustained activation of ERK compared to effects observed with PMA alone (data not shown). In each case, total ERK protein remained constant throughout. In separate studies, co-administration of FP did not lead to alterations in PMA-mediated phosphorylation of JNK (c-Jun N-terminal kinase; data not shown). Thus, co-administration of FP with PMA induced multiple early perturbations in differentiation-related proteins and signaling pathways, including (a) antagonism of p21^{WAF1/CIP1} induction, (b) acceleration of pRb dephosphorylation, (c) cleavage pRb and p27^{KIP1}; and (d) a more pronounced and sustained activation of ERK1/2.

### EXAMPLE 6

### Functional Role of ERK Activation in PMA/FP-Mediated Apoptosis

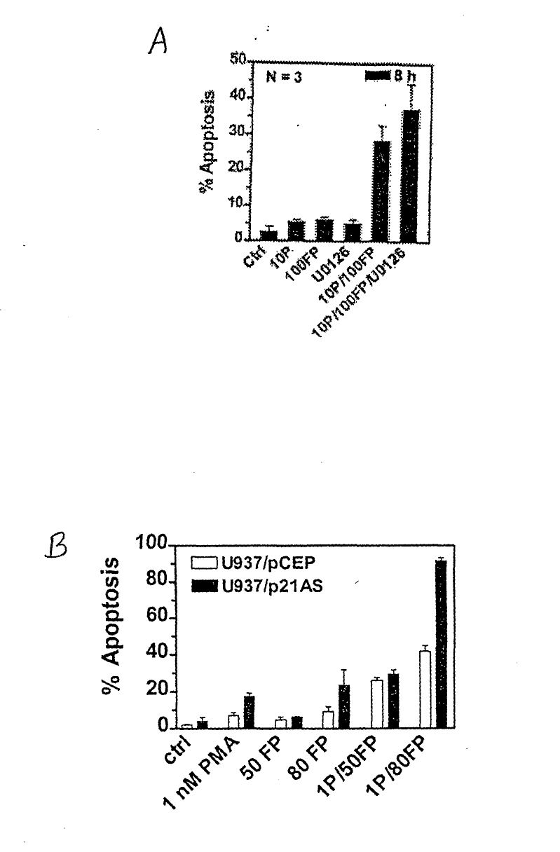

In view of evidence that stimulation of the ERK cascade can either promote [Zi and Agarwal, 1999] or inhibit [Xia et al. 1995] apoptosis, depending upon the stimulus and cell type, attempts were made to define the functional significance of enhanced ERK activation in cells treated with the PMA/FP combination (FIG. 6A). Consistent with the previous results, 1 h exposure to PMA (10 nM) induced ERK activation and this effect was more pronounced in cells treated with PMA and FP (data not shown). In both cases, ERK activation was opposed by addition of specific MEK1 inhibitors PD90859 (25 μM) or, to an even greater extent, by U0126 (25 μM), which has a higher affinity for the ATP binding site of MEK1 than PD98059 [Favata et al. 1998]. Addition of U0126 slightly increased PMA/FP-induced apoptosis from 28% to 37% after 8 h of co-treatment (FIG. 6A), arguing against the possibility that enhanced activation of the MEK1/ERK pathway is responsible for potentiation of PMA-induced apoptosis by FP.

### EXAMPLE 7

### Functional Role of Diminished p21^{WAF1/CIP1} Expression in PMA/FP-Mediated Apoptosis

Attempts were made to define the functional role of diminished p21^{WAF1/CIP1} expression in PMA/FP-mediated apoptosis using a U937 transfectant cell line stably expressing p21^{WAF1/CIP1} in the antisense configuration (i.e., U937/p21AS) [Wang et al. 1998]. It was hypothesized that if disruption of p21^{WAF1/CIP1} expression by FP contributes functionally to PMA/FP-mediated apoptosis, U937/p21AS cells would be less susceptible to potentiation of apoptosis because induction of p21^{WAF1/CIP1} is already impaired. To evaluate this possibility, U937/p21AS cells and their empty-vector counterparts (i.e., U937/pREP4) were exposed to FP in conjunction with a low concentration of PMA (1 nM). The latter concentration was selected to limit the extent of PMA-mediated apoptosis in U937/p21AS cells [Wang et al. 1998]. Following exposure to 1 nM PMA and 50 nM FP, enhancement of apoptosis was equivalent in U937/p21AS (29% apoptotic) and empty vector controls (26% apoptotic) as shown in FIG. 6B. However, when cells were exposed to 1 nM PMA and 80 nM FP, the extent of apoptosis was clearly more pronounced in U937/p21AS cells compared to empty-vector controls, 92% versus 42%, respectively (p<0.001).

While these findings do not rule out the possibility that FP-mediated dysregulation of p21^{WAF1/CIP1} contributes to the increase in cell death following PMA exposure, they do suggest that other factors play a role in the enhanced lethality of the PMA/FP combination.

### EXAMPLE 8

### Effects of PMA and FP on E2F/pRb Binding

Induction of leukemic cell differentiation by PMA requires CDKI induction, pRb dephosphorylation, and inactivation of E2F transcription factors via binding to the underphosphorylated form of pRB [Chellapan et al. 1991]. Therefore, the effects of FP co-administration were examined in relation to the interaction between E2F and pRb (data not shown).U937 cells were exposed to 10 nM PMA, 100 nM FP, or the combination for 8 h, after which pRb immunoprecipitates were obtained and treated with deoxycholate to dissociate pRb/E2F complexes. The E2F liberated from pRB after IP was evaluated by EMSA. U937 cells exposed to the combination of FP and PMA, or FP alone, displayed a clear reduction in E2F binding to labeled probe compared to PMA-treated cells, indicating that FP diminishes pRb/E2F binding. Furthermore, EMSA analysis of extracts from FP/PMA-treated cells displayed increased levels of a rapidly migrating E2F species, consistent with the preceding observations (data not shown).

Together, these findings raise the possibility that FP-mediated perturbations in pRb/E2F binding in PMA-treated cells may contribute to the lethal actions of this drug combination.

### EXAMPLE 9

### Co-Treatment with FP and SB Results in a Marked Apoptotic Response in U937 Human Leukemia Cells

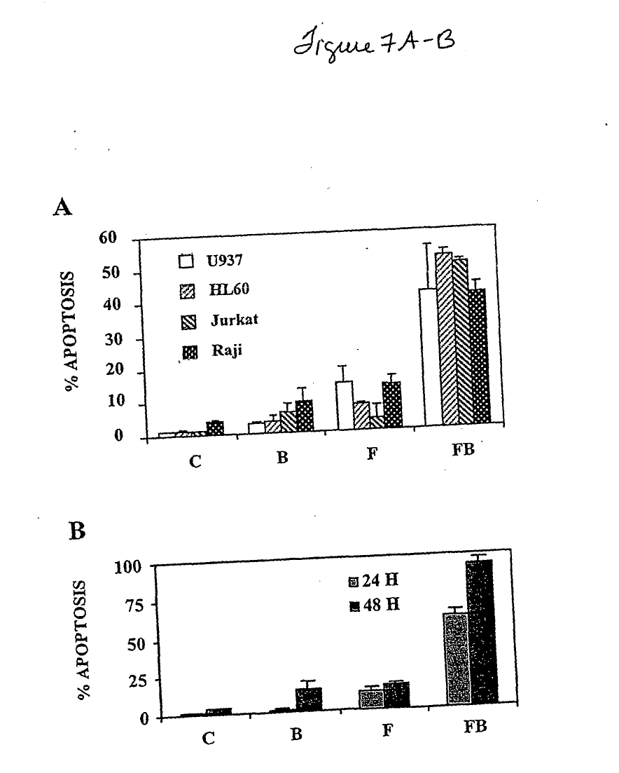

To characterize interactions between these agents, cells were exposed for 24 or 48 hr to 1 mM SB, 100 nM FP, or the combination, after which the extent of apoptosis was monitored. SB or FP alone were minimally toxic at 24 h or 48 h, inducing apoptosis in ≦15% of cells. In marked contrast, simultaneous exposure of U937 cells to these agents dramatically increased apoptosis (e.g., to ˜60% at 24 h and nearly 100% at 48 h; FIG. 7A). Parallel results were obtained when apoptosis was monitored by annexin/PI (data not shown). Induction of apoptosis by SB (1 mM)+FP (100 nM; 24 hr each) was then examined in other human leukemia cell lines (e.g., HL-60 promyelocytic, Jurkat T-lymphoblastic, and Raji B-lymphoblastic (FIG. 7B). In each case, a marked increase in lethality was observed in cells exposed to both agents, indicating that synergistic potentiation of SB-mediated apoptosis by FP is neither lineage- nor cell-type specific. Western blotting analyses showed that, consistent with these results, activation of procaspase-3, manifested by cleavage/degradation of the full-length Mr 32,000 species, was more pronounced in FP/SB treated U937 cells at 12 and 24 hours compared to the effects of either agent administered alone. Similarly, degradation of the caspase-3 substrate, PARP, from a Mr 115,000 full-length species to a Mr 85,000 cleavage product was occurred in parallel.

### EXAMPLE 10

### FP/SB-Mediated Apoptosis Primarily Involves the Intrinsic Pathway

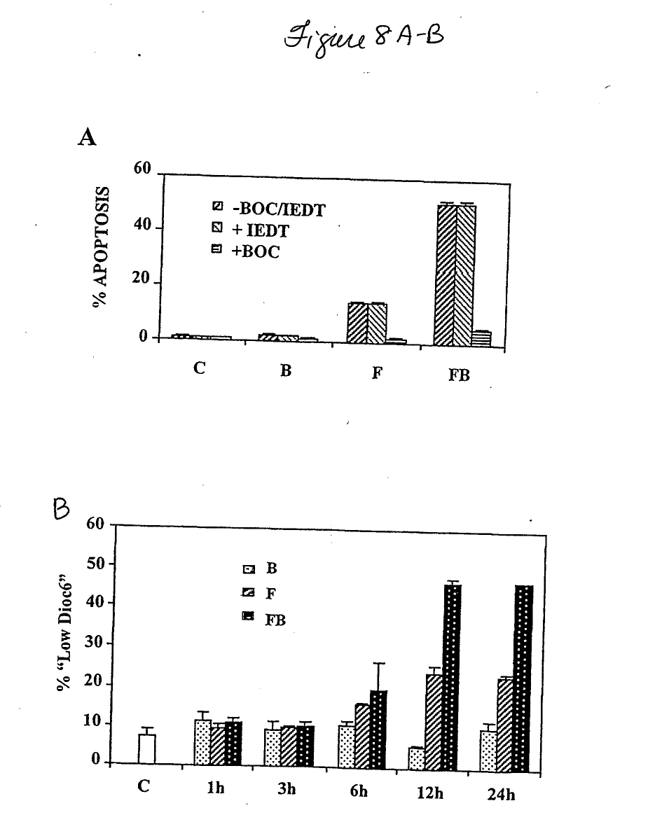

Treatment of U937 cells with the pan-caspase inhibitor BOC-fmk blocked FP/SB-induced apoptosis after 12 hours of exposure, whereas no inhibition was observed when the caspase-8 inhibitor IEDT-fmk was employed (FIG. 8A). Procaspase-8 activation, reflected by the appearance of a cleavage fragment, became clearly evident as early as 6 hr after treatment with FP/SB, an event followed by a decrease in levels of the caspase-8 substrate Bid, as demonstrated by Western blotting. Cleavage of caspase-8 and Bid were blocked by both the caspase-8 inhibitor IEDT-fmk or the pan-caspase inhibitor BOC-fmk. Together, these findings suggest that activation of the extrinsic, caspase-8-dependent pathway represents a secondary event in cells exposed to the combination of SB and FP.

### EXAMPLE 11

### Enhanced Apoptosis in U937 Cells Exposed to FP/SB is Associated with Loss of Mitochrondrial Membrane Potential (Δψₘ) and Cytochrome c Release

Early mitochondrial dysfunction has been shown to play important role in apoptotic events. To determine what effect exposure of cells to SB and FP might have on mitochondrial perturbations accompanying (or responsible for) apoptosis, cytoplasmic cytochrome c release and uptake of the lipophilic fluorochrome DiOC₆, reflecting maintenance of mitochondrial membrane potential (Δψₘ), were monitored. Cells exposed to 100 nM FP alone displayed a modest increase in the percentage of cells displaying a loss of Δψₘ at 12 or 24 hr. However, co-exposure of cells to 1 mM SB, which by itself exerted no effect on Δψₘ, resulted in a marked increase in mitochondrial discharge FIG. 8B). Moreover, cytochrome c release, which increased modestly as early as 1 hr after treatment with SB or FP alone, became very pronounced in cells exposed to both drugs for intervals ≧6 hr. These findings indicate that combined treatment of U937 cells with SB and FP is associated with early potentiation of mitochondrial injury.

### EXAMPLE 12

### U937 Cells Display an Impaired G₀/G₁ Arrest and Maturation Program Following Treatment with FP/SB

Flavopiridol is a potent CDK inhibitor (reviewed in Sedlacek, 2001) and arrests cells in different phases of the cell cycle, depending of the cell type (Shapiro et al., 1999; Sedlacek, 2001). Sodium butyrate is also known to induce cell cycle arrest Rosato et al., 2001). To determine what effect combined treatment with SB and FP would have on cell cycle traverse, the cell cycle distribution of U937 cells was examined 24 h and 48 h after administration of 1 mM SB, 100 nM FP or the combination. SB-treated cells displayed a prominent increase in the percentage of cells arresting in G₀/G₁ (e.g 48.1% and 81.6% at 24 and 48 h respectively). Arrest in G₀/G₁ following SB treatment was accompanied by increased expression of the differentiation marker CD11b. FP-treated cells also exhibited an increase in the G₀/G₁ population at the 48-hr interval (i.e., ˜53%; although in contrast to SB, this was not accompanied by an increase in CD11b expression. However, when cells were treated with the combination of FP and SB, the majority of cells appeared in the subdiploid fraction at both 24 and 48 hr, thus preventing accumulation of cells in G₀/G₁, Furthermore, FP co-administration antagonized SB-induced differentiation, manifested by a marked reduction the percentage CD11b-expressing cells.

### EXAMPLE 13

### Co-Exposure to FP Blocks SB-Induced Expression of the CDK-Inhibitor p21^{WAF1/CIP1}

Previous studies have demonstrated the importance of the CDKI p21^{WAF1/CIP1} in leukemic cell maturation, including that induced by SB. Therefore, the effects of FP on SB-mediated induction of p21^{WAF1/CIP1} was examined. Administration of 1 mM SB induced a time-dependent increase in levels of p21^{WAF1/CIP1}, which was first detectable at 1-3 hr, and which was very robust at exposure intervals≧12 hr. FP alone did not modify p21^{WAF1/CIP1} expression. However, co-administration of FP essentially abrogated induction of p21^{WAF1/CIP1} by SB at each interval examined. This effect was not prevented by the the pan-caspase inhibitor B-D-fmk (20 μM), indicating that FP interferes with p21^{WAF1/CIP1} induction through a caspase-independent mechanism. Such results are concordant with the possibility that FP may act in a generalized way to block p21^{WAF1/CIP1} induction by different classes of differentiation-inducers.

In contrast to p21^{WAF1/CIP1}, the CDKI p27^{KIP1} was expressed basally, but levels were minimally affected by treatment of cells with SB or PMA alone. However, in cells exposed to the combination of SB and FP, a rapidly migrating 23,000 Mᵣ fragment became apparent at 12 hr, and was quite pronounced by 24 hr, events accompanied by reduced levels of the full-length p27^{KIP1} protein. Also in contrast to p21^{WAF1/CIP1}, cleavage of p27^{KIP1} in FP/SB-treated cells was prevented by B-D-fmk (20 μM), indicating that this event represented a secondary process downstream of caspase activation.

### EXAMPLE 14

### The Extent of SB-Induced Acetylation of Histones H3 and H4 is Not Modified by Co-Administration FP

There is considerable evidence implicating acetylation and deacetylation of histones in the regulation of transcription (n20, n41,-43, n45 Marks et al. 2000). In this regard, several classes of histone deacetylase inhibitors (HDIs) have been described (Marks et al., 2000), several of which, including SB, act as potent differentiation inducers, (Kouzarides, 1999; Wang X M et al., 1998 (RR:55); Janson et al., 1997 (RR:54). To investigate whether the response of U937 cells to FP/SB might involve modulation of histone acetylation, acetylated histones-3 (H3) and -4 (H4) were monitored at various intervals following exposure of cells to 1 mM SB, 100 nM FP or the combination. Acetylation of H3 and H4 was observed in SB treated cells and this acetylation was not demonstrably modified by co-treatment with FP. Such findings argue against the possibility that the observed effects of the combination of FP and SB on leukemic cell maturation and apoptosis result from alterations in the extent of histone acetylation.

### EXAMPLE 15

### The Combination of FP and SB Induces Changes in the Levels of Antiapoptotic Proteins Through Both Caspase-Dependent and -Independent Mechanisms

The Bcl-2 family of apoptotic proteins contains members that promote survival (e.g., Bcl-2, Bcl-X_{L}, etc.) whereas others exert pro-apoptotic actions (Bak, Bax, etc.). Attempts were therefore made to determine what effect combined treatment with SB and FP might have on levels of these proteins. In cells exposed to either SB or FP, a rapidly migrating Bcl-2 species could be faintly discerned at 12 and 24 hr, presumably corresponding to a pro-apoptotic cleavage product. However, cleavage of Bcl-2 was considerably more pronounced in cells exposed to both agents. Co-administration of B-D-fmk blocked this event, consistent with the notion that this species represents a Bcl-2 degradation product.

Recently, FP has been reported to induce a concentration-dependent reduction in expression of the anti-apoptotic Bcl-2 family member Mcl-1 in B-CLL cells (Kitada et. al, 2000). When U937 cells were exposed to 100 nM FP, reductions in levels of Mcl-1 were noted at 12 and 24 hours, whereas SB exerted minimal effects. However, the combination of FP and SB dramatically reduced Mcl-1 expression. Moreover, as previously observed in CLL cells exposed to FP alone, {Kitada et al. (2000), and in contrast to results involving Bcl-2, this event was not antagonized by co-administration of B-D-fmk. Such findings indicate that SB and FP cooperate to diminish Mcl-1 expression through a caspase-independent mechanism.

FP as well as the combination of FP and SB resulted in modest decreases in levels of the anti-apoptotic protein XIAP (X-inhibitor of apoptosis), an effect that was not blocked by B-D-fmk. On the other hand, neither SB, FP, nor the combination altered expression of the pro-apoptotic proteins Bax and Bak. Taken together, these findings raise the possibility that the combination of FP and SB may induce apoptosis, at least in part, by reducing levels of certain anti-apoptotic proteins. Furthermore, they suggest that such actions may proceed through both caspase-dependent and caspase-independent mechanisms.

### EXAMPLE 16

### FP/SB Co-Administration Results in pRB Cleavage

As noted above, both FP and SB can modulate cell cycle progression, a process that is also regulated the pRb/E2F axis (rev. in Fan & Steer, 2000; Studzinski & Harrison, 1999). Therefore, attempts were made to assess the effects of this drug combination on pRb phosphorylation status. Whereas FP (100 nM) by itself did not affect levels of dephosphorylated pRb at 12 or 24 hr, SB (1 mM), in marked contrast, induced a substantial increase in levels of the underphosphorylated form at 24 hr. Co-exposure to FP/SB resulted in a marked decrease in levels of full-length underphosphorylated pRb, accompanied by a clearly discernible 65 kDa species at both 12 and 24 hr, presumably corresponding to a pRb cleavage product. This was confirmed by the observed reduction in levels of total pRb, as well as by the ability of the caspase inhibitor Boc-fmk to block formation of the fragment.

FP is known to inhibit the activities of cyclin-dependent kinases involved in pRb phosphorylation, notably cdk2 and cdk4 (Carlson et al., 1999). Western blot analysis was performed to evaluate the effects of this drug combination on. Our results show that FP alone was able to induce a moderate inhibition of both cdks' activities, meanwhile FP/SB combination provoked a drastic reduction on the amount of cdk2/4-phosphorylated pRb that was only moderately reverted by the caspase inhibitor B-D-FMK. Changes in activity of both cdks were confirmed by the fact that their levels of expression were not modified at any time or treatment.

In its hypophosphorylated form, pRb binds to members of the E2F transcription factor family, thereby inhibiting E2F-1-mediated G₁S progression (rev. in Dyson, 1998). Therefore, the effects of FP/SB co-treatment were examined in relation to E2F-1/pRb interactions. Following exposure to the agents alone or in combination for 8 hours, pRb immunoprecipitates were obtained and treated with deoxycholate to dissociate pRb/E2F complexes. E2F liberated from pRb immunoprecipitates was then evaluated by EMSA. Cells exposed to the combination of FP and SB displayed a marked reduction in E2F binding to labeled probe, reflecting a diminished pRb/E2F association. In contrast, Western blot analysis demonstrated the absence of changes in total E2F1 expression following any treatment. Analysis of the pRb family members (pRB, p130 and p107) demonstrated that levels of p130 and p107, as observed in the case of pRb, were substantially reduced in extracts obtained from cells exposed to both FB and SB. Together, these findings indicate co-exposure of leukemic cells to FB and SB results in marked perturbations in the pRb/E2F-1 axis.

### EXAMPLE 17

### FP/SB Treatment Induces Down-Regulation of Cyclins-D1, -E and -A

Cells traversing the cell cycle are regulated by cyclin/cdk complexes. Progression through G₁ and into S phase requires the sequential expression of cyclins D, E and A (rev. in Studzinski & Harrison, 1999). Both FP and SB have been reported to exert critical regulatory effects on cyclin expression (Carlson et al., 1996, 1999; Lee, Chang, Tebalt, et al., 1999). A dose-dependent reduction in cyclin D1 levels was observed in cells treated with FP which was first observed at 100 nM and which was virtually complete at 300 nM after 12 and 24 hours treatment. Moreover, SB alone (1 mM) induced a moderate decrease of cyclin D1 that was mainly observed after 24 hours of exposure. However, the combination of FP and SB resulted in essentially the total disappearance of cyclin D1 protein after only 12 hours of co-treatment. Similarly, co-exposure to FP/SB substantially diminished levels of cyclin E and, to a slightly lesser extent, cyclin A. Thus, co-treatment of U937 cells with FP/SB resulted in profound reductioins in expression of various cyclins involved in cell cycle progression through G1₁S.

### EXAMPLE 18

### Blasts from AML Patients are Sensitive to FP/SB-Mediated Apoptosis

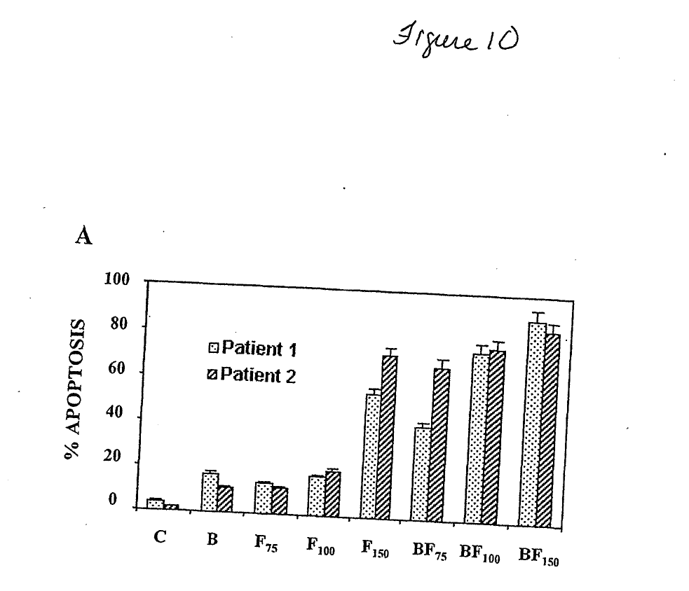

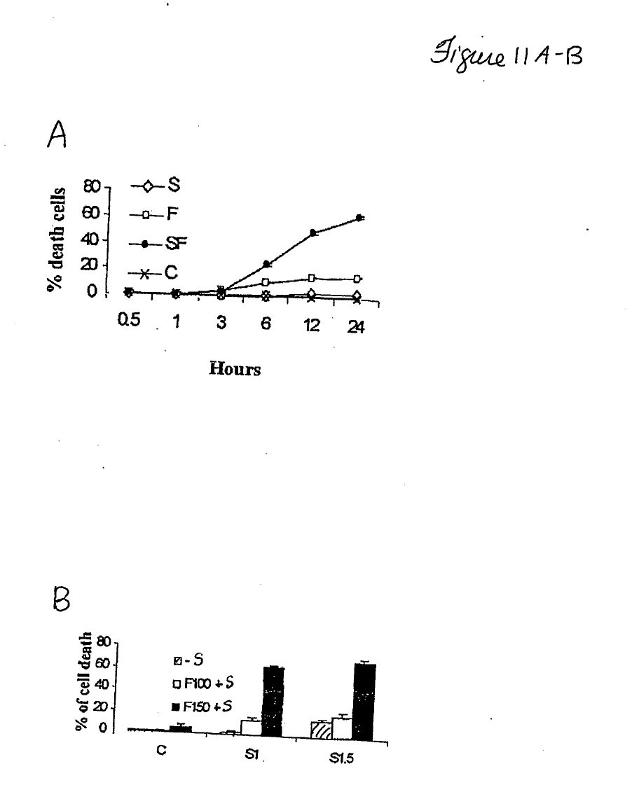

To determine whether primaRy AML cells might mimic the responses of continuously cultured cell lines such as U937 and HL-60 to these agents, leukemic blasts obtained from the peripheral blood of two patients with refractory AML were isolated and exposed to various concentrations of FP and SB alone or in combination for 24 hours (FIG. 10). Both 75 nM or 100 nM FP and 1 mM SB alone induced a modest degree of apoptosis (e.g. ˜15-20%) in the two samples. However, when cells were exposed to both 100 nM FP and 1 mM SB, the extent of apoptosis increased dramatically (i.e., to ˜80%). Western blot analysis of blasts obtained from patient #1 displayed changes similar to those observed in the case of U937 cells (FIG. 11B). Specifically, co-administration of FP and SB resulted in a marked increase in cleavage of procaspase-3, Bcl-2, and pRb, but no changes in levels of Bax. Similar results were obtained in blasts obtained from patient #2 (data not shown). Thus, these findings indicate that combined treatment with SB and FP results in a marked increase in caspase activation and apoptosis in primary AML blast specimens, analogous to findings in continuously cultured leukemic cell lines.

### EXAMPLE 19

### Co-Treatment with FP and the Histone Deacetylase Inhibitor Suberoylanilide Hydroxamic Acid (SAHA) Results in a Marked Apoptotic Response in U937 and HL60 Human Leukemia Cells

To characterize interactions between FP and a the histone deactylase inhibitor SAHA, U937 myelomonocytic human leukemia cells were exposed to SAHA alone, or the combination of SAHA with 100 nM FP. As can be seen in FIG. 11A, SAHA alone was ineffective for inducing cell death and FP alone induced only a modest level of cell death. However, the combination of the two agents resulted in better than 60% cell death 24 hours after administration. Experiments carried out with HL-60 promyelocytic leukemia cells showed similar results. HL-60 cells were exposed to SAHA at 1 and 1.5 μM alone or in combination with 100 or 150 nM FP. SAHA alone produced only modest levels of cell death. However, in combination with either 100 or 150 nM FP, cell death percentages rose to greater than 60%. This example illustrates that a second example of a histone deacetylase inhibitor is also effective in the practice of the present invention.

### EXAMPLE 20

### Enhanced Apoptosis in U937 Cells Exposed to FP/SAHA is Associated with Loss of Mitochrondrial Membrane Potential (Δψₘ) and Cytochrome c Release

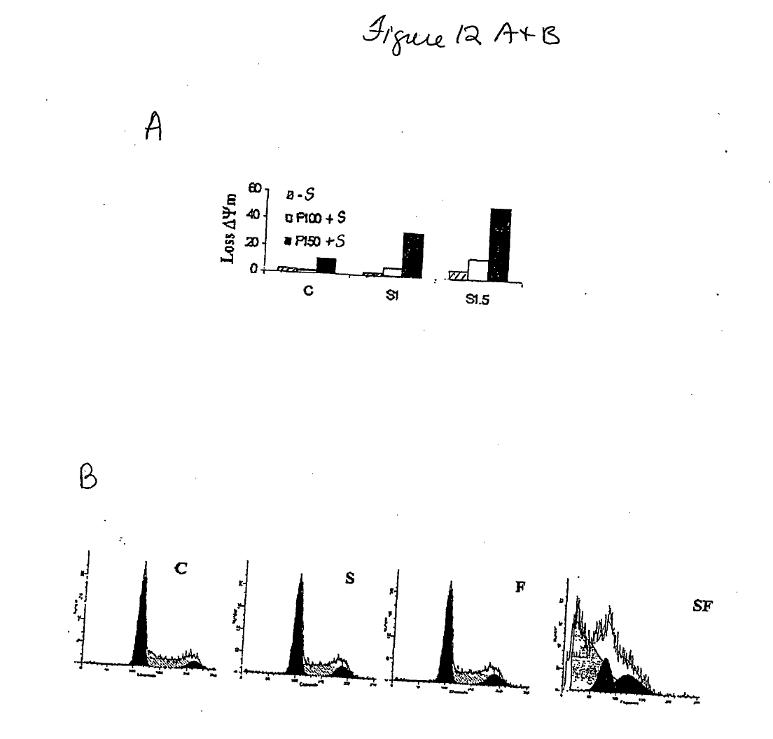

To determine what effect exposure of cells to SAHA and FP might have on mitochondrial perturbations accompanying (or responsible for) apoptosis, cytoplasmic cytochrome c release and uptake of the lipophilic fluorochrome DiOC₆, reflecting maintenance of mitochondrial membrane potential (Δψₘ), were monitored. The results are depicted in FIG. 12A and demonstrate that co-exposure of cells to either 1 and 1.5 μM SAHA and FP, which by itself exerted no effect on Δψₘ, resulted in a marked increase in mitochondrial discharge. These findings indicate that combined treatment of HL60 cells with SAHA and FP is associated with early potentiation of mitochondrial injury.

### EXAMPLE 21

### SAHA/FP Combination Produces Disruption in Cell Cycle

To determine what effect combined treatment with SAHA and FP would have on cell cycle traverse, the cell cycle distribution of U937 cells was examined 24 h after administration of SAHA, FP or the combination of both. SAHA-treated cells displayed a prominent increase in the percentage of cells arresting in G₀/G₁. FP-treated cells also exhibited an increase in the G₀/G₁ population. However, when cells were treated with the combination of FP and SAHA, the majority of cells appeared in the subdiploid fraction, thus preventing accumulation of cells in G₀/G₁,

### EXAMPLE 22

### Flavopiridol in Combination with Four Different Histone Deacetylase Inhibitors

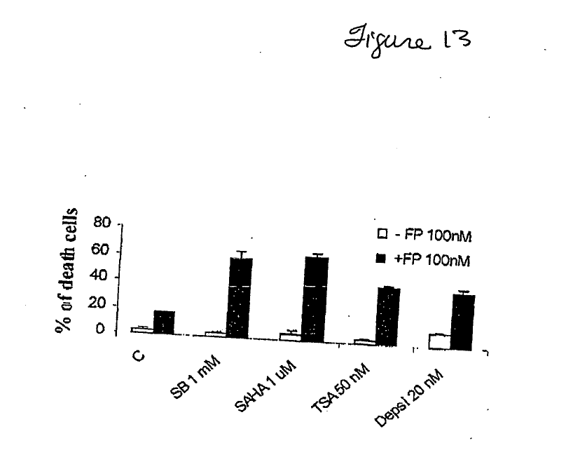

The effect of cell death of four different histone deacetylase inhibitors in combination with FP was assessed. As can be seen in FIG. 13, in each case, the combination of histone deacetylase inhibitor and FP was far more efficacious in inducing cell death than FP alone, suggesting that the increase in cell death observed by combining a histone deacetylase inhibitor with a differentiation agent is a general phenomenon.

### EXAMPLE 23

### Co-Treatment with FP and All Trans Retinoic Acid (ATRA) Results in a Marked Apoptotic Response and Loss of Mitochrondrial Membrane Potential in HL60 Human Leukemia Cells

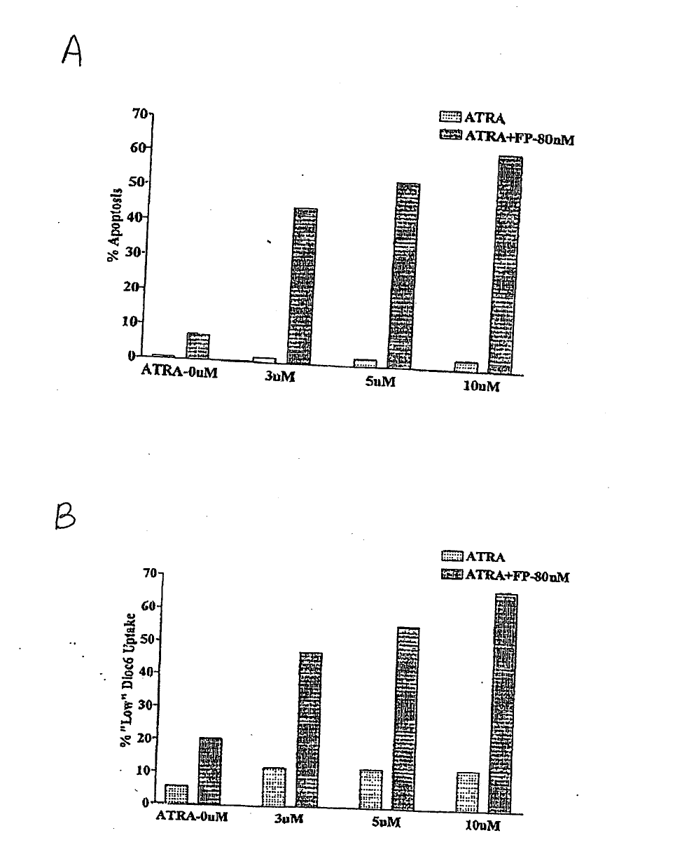

The effect of the combination of the retinol ATRA and FP on the % apoptosis induced in HL60 cells as assessed. The results are shown in FIG. 14A. As can be seen, 3, 5, or 10 μM ATRA alone or FP alone had a minimal effect on apoptosis. However, when 3, 5, or 10 μM ATRA is combined with 80 nM FP, % apoptosis increased sharply to between about 50 to over 60%.

The effect of co-administration of the two agents on mitochondrial membrane potential (Δψₘ), was also monitored. The results are depicted in FIG. 14B and demonstrate that co-exposure of cells to either 3, 5, or 10 μM ATRA and 80 nM FP, which by itself exerted no effect on Δψₘ, resulted in a marked increase in mitochondrial discharge. These findings indicate that combined treatment of HL60 cells with ATRA and FP is associated with early potentiation of mitochondrial injury.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Bagchi, S., Raychaudhuri, P., and Nevins, J. R. Adenovirus E1A proteins can dissociate heteromeric complexes involving the E2F transcription factor: a novel mechanism for E1A trans-activation. Cell, 62: 659-669, 1990.
Bible, K. C. and Kaufmann, S. H. Cytotoxic synergy between flavopiridol (NSC 649890, L86-8275) and various antineoplastic agents: the importance of sequence of administration. Cancer Res., 57: 3375-3380, 1997.
Carey, J. O., Posekany, K. J., deVente, J. E., Pettit, G. R., and Ways, D. K. Phorbol ester-stimulated phosphorylation of PU.1: association with leukemic cell growth inhibition. Blood, 87: 4316-4324, 1996.
Carlson, B., Lahusen, T., Singh, S., Loaiza-Perez, A., Worland, P. J., Pestell, R., Albanese, C., Sausville, E. A., and Senderowicz, A. M. Down-regulation of cyclin D1 by transcriptional repression in MCF-7 human breast carcinoma cells induced by flavopiridol. Cancer Res., 59: 4634-4641, 1999.
Carlson B, Lahusen T, Singh S, Loaiza-Perez A, Worland P J, Pestell R, Albanese C, Sausville E A, Senderowicz A M. Down-regulation of cyclin D1 by transcriptional repression in MCF-7 human breast carcinoma cells induced by flavopiridol. Cancer Res Sep. 15, 1999;59(18):4634-41
Chellapan, S. P., Hiebert, S., Mudryj, M., Horowitz, J. M., and Nevins, J. R. The E2F transcription factor is a cellular target for the RB protein. Cell, 65:1053-1061, 1991.
Chinery, R., Brockman, J. A., Peeler, M. O., Shyr, Y., Beauchamp, R. D., and Coffey, R. J. Antioxidants enhance the cytotoxicity of chemotherapeutic agents in colorectal cancer: a p53-independent induction of p21WAF1/CIP1 via C/EBPbeta. Nat. Med., 3: 1233-1241, 1997.
Chou, T. C. and Talalay, P. Quantitative analysis of dose-effect relationships: the combined effects of multiple drugs or enzyme inhibitors. Adv. Enzyme Regul., 22: 27-55, 1984.
Christian, M. C., Pluda, J. M., Ho, P. T., Arbuck, S. G., Murgo, A. J., and Sausville, E. A. Promising new agents under development by the Division of Cancer Treatment, Diagnosis, and Centers of the National Cancer Institute. Semin. Oncol., 24: 219-240, 1997.
Dyson N. The regulation of E2F by pRB-family proteins. Genes Dev Aug. 1, 1998;12(15):2245-62.
Eymin, B., Haugg, M., Droin, N., Sordet, O., Dimanche-Boitrel, M. T., and Solary, E. p27Kip1 induces drug resistance by preventing apoptosis upstream of cytochrome c release and procaspase-3 activation in leukemic cells. Oncogene, 18: 1411-1418, 1999.
Fan, J. and Bertino, J. R. K-ras modulates the cell cycle via both positive and negative regulatory pathways. Oncogene, 14: 2595-2607, 1997.
Fan G., Steer C. J. The role of retinoblastoma protein in apoptosis. Apoptosis, 4: 21-29, 2000.
Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., Copeland, R. A., Magolda, R. L., Scherle, P. A., and Trzaskos, J. M. Identification of a novel inhibitor of mitogen-activated protein kinase kinase. J. Biol. Chem., 273: 18623-18632, 1998.
Freemerman, A. J., Vrana, J. A., Tombes, R. M., Jiang, H., Chellappan, S. P., Fisher, P. B., and Grant, S. Effects of antisense p21 (WAF1/CIP1/MDA6) expression on the induction of differentiation and drug-mediated apoptosis in human myeloid leukemia cells (HL-60). Leukemia, 11: 504-513, 1997.
Gunji, H., Hass, R., and Kufe, D. Internucleosomal DNA fragmentation during phorbol ester-induced monocytic differentiation and G0/G1 arrest. J. Clin. Invest, 89: 954-960, 1992. An, B. and Dou, Q. P. Cleavage of retinoblastoma protein during apoptosis: an interleukin 1 beta-converting enzyme-like protease as candidate. Cancer Res., 56: 438-442, 1996.
Hoffman, B. and Liebermann, D. A. Molecular controls of apoptosis: differentiation/growth arrest primary response genes, proto-oncogenes, and tumor suppressor genes as positive & negative modulators. Oncogene, 9: 1807-1812, 1994.
Grant, S., Jarvis, W. D., Swerdlow, P. S., Turner, A. J., Traylor, R. S., Wallace, H. J., Lin, P. S., Pettit, G. R., and Gewirtz, D. A. Potentiation of the activity of 1-beta-D-arabinofuranosylcytosine by the protein kinase C activator bryostatin 1 in HL-60 cells: association with enhanced fragmentation of mature DNA. Cancer Res., 52: 6270-6278, 1992.
Janson W, Brandner G and Siegel J: Butyrate modulates DNA-damage-induced p53 response by induction of p53-independent differentiation and apoptosis. Oncogene 15: 1395-1406, 1997.
Jiang, H., Lin, J., Su, Z. Z., Collart, F. R., Huberman, E., and Fisher, P. B. Induction of differentiation in human promyelocytic HL-60 leukemia cells activates p21, WAF1/CIP1, expression in the absence of p53. Oncogene, 9: 3397-3406, 1994.
Johnson, D. G. and Walker, C. L. Cyclins and cell cycle checkpoints. Annu. Rev. Pharmacol. Toxicol., 39: 295-312, 1999.
Kitada S, Zapata J M, Andreeff M, Reed J C. Protein kinase inhibitors flavopiridol and 7-hydroxy-staurosporine down-regulate antiapoptosis proteins in B-cell chronic lymphocytic leukemia. Blood. Jul. 15, 2000;96(2):393-7.
Kouzarides T. Histone acetylases and deacetylases in cell proliferation. Curr Opin Genet Dev 1999;9:40-8.
Marks P A, Richon V M, Rifkind R A. Histone deacetylase inhibitors: inducers of differentiation or apoptosis of transformed cells. J Natl Cancer Inst. Aug. 2, 2000;92(15):1210-6.
Lee H R, Chang T H, Tebalt M J 3rd, Senderowicz A M, Szabo E. Int J Oncol 1999 15: 161-6. Induction of differentiation accompanies inhibition of Cdk2 in a non-small cell lung cancer cell line.
Lee, H. R., Chang, T. H., Tebalt, M. J., III, Senderowicz, A. M., and Szabo, E. Induction of differentiation accompanies inhibition of Cdk2 in a non-small cell lung cancer cell line. Int. J. Oncol., 15: 161-166, 1999.
Li, P., Nijhawan, D., Budihardjo, I., Srinivasula, S. M., Ahmad, M., Alnemri, E. S., and Wang, X. Cytochrome c and dATP-dependent formation of Apaf-1/caspase-9 complex initiates an apoptotic protease cascade. Cell, 91: 479-489, 1997.
Loubat, A., Rochet, N., Turchi, L., Rezzonico, R., Far, D. F., Auberger, P., Rossi, B., and Ponzio, G. Evidence for a p23 caspase-cleaved form of p27[KIP1] involved in G1 growth arrest. Oncogene, 18: 3324-3333, 1999.
Lundberg, A. S. and Weinberg, R. A. Control of the cell cycle and apoptosis. Eur. J. Cancer, 35: 1886-1894, 1999.
Marchetti, P., Susin, S. A., Decaudin, D., Gamen, S., Castedo, M., Hirsch, T., Zamzami, N., Naval, J., Senik, A., and Kroemer, G. Apoptosis-associated derangement of mitochondrial function in cells lacking mitochondrial DNA. Cancer Res., 56: 2033-2038, 1996.
McIntyre A, Gibson P R, Young G P. Butyrate production from dietary fibre and protection against large bowel cancer in a rat model. Gut March 1993;34(3):386-391.
Pellizzaro C, Coradini D, Daniotti A, Abolafio G, Daidone M G. Modulation of cell cycle-related protein expression by sodium butyrate in human non-small cell lung cancer cell lines. Int J Cancer Mar. 1, 2001;91(5):654-7
McIntyre A, Young G P, Taranto T, Gibson P R, Ward P B. Different fibers have different regional effects on luminal contents of rat colon. Gastroenterology November 1991;101(5):1274-81
Meikrantz, W. and Schlegel, R. Apoptosis and the cell cycle. J. Cell Biochem., 58: 160-174, 1995.
Parker, B. W., Kaur, G., Nieves-Neira, W., Taimi, M., Kohlhagen, G., Shimizu, T., Losiewicz, M. D., Pommier, Y., Sausville, E. A., and Senderowicz, A. M. Early induction of apoptosis in hematopoietic cell lines after exposure to flavopiridol. Blood, 91: 458-465, 1998.
Pines, J. Cyclins and cyclin-dependent kinases: theme and variations. Adv. Cancer Res., 66: 181-212, 1995.
Powell, B. L., Wang, L. M., Gregory, B. W., Case, L. D., and Kucera, G. L. GM-CSF and asparaginase potentiate ara-C cytotoxicity in HL-60 cells. Leukemia, 9: 405-409, 1995.
Rosato R R, Wang Z, Gopalkrishnan R V, Fisher P B, Grant S. Evidence of a functional role for the cyclin-dependent kinase-inhibitor p21WAF1/CIP1/MDA6 in promoting differentiation and preventing mitochondrial dysfunction and apoptosis induced by sodium butyrate in human myelomonocytic leukemia cells (U937). Int J Oncol July 2001;19(1):181-.
Rosenthal, N. Identification of regulatory elements of cloned genes with functional assays. Methods Enzymol., 152: 704-720, 1987.
Ruan, S., Okcu, M. F., Ren, J. P., Chiao, P., Andreeff, M., Levin, V., and Zhang, W. Overexpressed WAF1/Cip1 renders glioblastoma cells resistant to chemotherapy agents 1,3-bis(2-chloroethyl)-1-nitrosourea and cisplatin. Cancer Res., 58: 1538-1543, 1998.
Sedlacek H H. Mechanisms of action of flavopiridol. Crit Rev Oncol Hematol. May 2001;38(2):139-70.
Senderowicz, A. M., Headlee, D., Stinson, S. F., Lush, R. M., Kalil, N., Villalba, L., Hill, K., Steinberg, S. M., Figg, W. D., Tompkins, A., Arbuck, S. G., and Sausville, E. A. Phase I trial of continuous infusion flavopiridol, a novel cyclin-dependent kinase inhibitor, in patients with refractory neoplasms. J. Clin. Oncol., 16: 2986-2999, 1998.
Senderowicz, A. M. Flavopiridol: the first cyclin-dependent kinase inhibitor in human clinical trials. Invest. New Drugs, 17: 313-320, 1999.
Shapiro G I, Koestner D A, Matranga C B, Rollins B J. Flavopiridol induces cell cycle arrest and p53-independent apoptosis in non-small cell lung cancer cell lines. Clin Cancer Res. October 1999;5(10):2925-38.
Sherr, C. J. and Roberts, J. M. Inhibitors of mammalian G1 cyclin-dependent kinases. Genes Dev., 9: 1149-1163, 1995
Single, B., Leist, M. and Nicotera, P. Simultaneous release of adenylate kinase and cytochrome c in cell death. Cell Death Differ., 5:1001-1003, 1998.
Sordet, O., Bettaieb, A., Bruey, J. M., Eymin, B., Droin, N., Ivarsson, M., Garrido, C., and Solary, E. Selective inhibition of apoptosis by TPA-induced differentiation of U937 leukemic cells. Cell Death Differ., 6: 351-361, 1999.
Studzinski G P, Harrison L E. Int Rev Cytol 1999;189:1-58. Differentiation-related changes in the cell cycle traverse.
Suzuki, A., Tsutomi, Y., Akahane, K., Araki, T., and Miura, M. Resistance to Fas-mediated apoptosis: activation of caspase 3 is regulated by cell cycle regulator p21WAF1 and IAP gene family ILP. Oncogene, 17: 931-939, 1998.
Tewari, M., Quan, L. T., O'Rourke, K., Desnoyers, S., Zeng, Z., Beidler, D. R., Poirier, G. G., Salvesen, G. S., and Dixit, V. M. Yama/CPP32 beta, a mammalian homolog of CED-3, is a CrmA-inhibitable protease that cleaves the death substrate poly(ADP-ribose) polymerase. Cell, 81: 801-809, 1995.
Trock B, Lanza E, Greenwald P. Dietary fiber, vegetables, and colon cancer: critical review and meta-analyses of the epidemiologic evidence. J Natl Cancer Inst Apr. 18, 1990;82(8):650-61
Vrana, J. A., Saunders, A. M., Chellappan, S. P., and Grant, S. Divergent effects of bryostatin 1 and phorbol myristate acetate on cell cycle arrest and maturation in human myelomonocytic leukemia cells (U937). Differentiation., 63: 33-42, 1998.
Wang Z, Van Tuyle G, Conrad D, Fisher P B, Dent P and Grant S: Dysregulation of the cyclin-dependent kinase inhibitor p21^{WAF1/CIP1/MDA6} increases the susceptibility of human leukemia cells (U937) to 1-beta-D-arabinofuranosylcytosine-mediated mitochondrial dysfunction and apoptosis. Cancer Res 15:1259-1267, 1999.
Wang, S., Vrana, J. A., Bartimole, T. M., Freemerman, A. J., Jarvis, W. D., Kramer, L. B., Krystal, G., Dent, P., and Grant, S. Agents that down-regulate or inhibit protein kinase C circumvent resistance to 1-beta-D-arabinofuranosylcytosine-induced apoptosis in human leukemia cells that overexpress Bcl-2. Mol. Pharmacol., 52: 1000-1009, 1997.
Wang X M, Wang X, Li J and Evers B M: Effects of 5-azacytidine and butyrate on differentiation and apoptosis of hepatic cancer cell lines. Ann Surg 227: 922-931, 1998.
Wang, Z., Su, Z. Z., Fisher, P. B., Wang, S., VanTuyle, G., and Grant, S. Evidence of a functional role for the cyclin-dependent kinase inhibitor p21(WAF1/CIP1/MDA6) in the reciprocal regulation of PKC activator-induced apoptosis and differentation in human myelomonocytic leukemia cells. Exp. Cell Res., 244: 105-116, 1998.
Wang, Z., Van Tuyle, G., Conrad, D., Fisher, P. B., Dent, P., and Grant, S. Dysregulation of the cyclin-dependent kinase inhibitor p21WAF1/CIP1/MDA6 increases the susceptibility of human leukemia cells (U937) to 1-beta-D-arabinofuranosylcytosine-mediated mitochondrial dysfunction and apoptosis. Cancer Res., 59: 1259-1267, 1999.
Weinberg, R. A. The retinoblastoma protein and cell cycle control. Cell, 81: 323-330, 1995.
Xia, Z., Dickens, M., Raingeaud, J., Davis, R. J., and Greenberg, M. E. Opposing effects of ERK and JNK-p38 MAP kinases on apoptosis. Science, 270:1326-1331, 1995.
Yee, A. S., Reichel, R., Kovesdi, I., and Nevins, J. R. Promoter interaction of the E1A-inducible factor E2F and its potential role in the formation of a multi-component complex. EMBO J., 6: 2061-2068, 1987.
Zamzami, N., Marchetti, P., Castedo, M., Decaudin, D., Macho, A., Hirsch, T., Susin, S. A., Petit, P. X., Mignotte, B., and Kroemer, G. Sequential reduction of mitochondrial transmembrane potential and generation of reactive oxygen species in early programmed cell death. J. Exp. Med., 182: 367-377, 1995.
Zhang, Y., and S. P. Chellappan. 1995. Cloning and characterization of human DP2, a novel dimerization partner of E2F. Oncogene 10:2085-2093.
Zhang, Y., Fujita, N., and Tsuruo, T. Caspase-mediated cleavage of p21Waf1/Cip1 converts cancer cells from growth arrest to undergoing apoptosis. Oncogene, 18: 1131-1138, 1999.
Zi, X. and Agarwal, R. Modulation of mitogen-activated protein kinase activation and cell cycle regulators by the potent skin cancer preventive agent silymarin. Biochem. Biophys. Res. Commun., 263: 528-536, 1999.

1. A method for promoting apoptosis in cancer cells, comprising
co-administering to said cancer cells to a cyclin dependent kinase inhibitor and an agent that induces cell differentiation, wherein said cyclin dependent kinase inhibitor and said agent that induces cell differentiation are present in a quantity sufficient to promote apoptosis of said cancer cells.

2. The method of claim 1 wherein said agent that induces cell differentiation is selected from the group consisting of histone deacetylation inhibitors, protein kinase C activators, retinoids, and Vitamin D3.

3. The method of claim 2 wherein said histone deacetylation inhibitor is selected from the group consisting of sodium butyrate, phenylbutyrate, suberoylanilide hydroxamic acid, depsipeptide, tricostatin A, MS-275, and CI-994.

4. The method of claim 2 wherein said protein kinase C activator is selected from the group consisting of PMA and bryostatin.

5. The method of claim 2 wherein said retinoid is all trans retinoic acid.

6. The method of claim 2 wherein said cyclin dependent kinase inhibitor is selected from the group consisting of flavopiridol, UCN-01, roscovitine, olomoucine, and butyrolactone.

7. The method of claim 1 wherein said cancer cells are selected from the group consisting of leukemia cells, prostate cancer cells, breast cancer cells, myeloma cells, and lymphoma cells.

8. A method for treating cancer by promoting apoptosis of cancer cells in a patient in need thereof, comprising
co-administering to said patient a cyclin dependent kinase inhibitor and an agent that induces cell differentiation, wherein said cyclin dependent kinase inhibitor and said agent that induces cell differentiation are administered in a quantity sufficient to promote apoptosis of said cancer cells.

9. The method of claim 8 wherein said agent that induces cell differentiation is selected from the group consisting of histone deacetylation inhibitors, protein kinase C activators, retinoids, and Vitamin D3.

10. The method of claim 9 wherein said histone deacetylation inhibitor is selected from the group consisting of sodium butyrate, phenylbutyrate, suberoylanilide hydroxamic acid, depsipeptide, tricostatin A, MS-275, and CI-994.

11. The method of claim 9 wherein said protein kinase C activator is selected from the group consisting of PMA and bryostatin.

12. The method of claim 9 wherein said retinoid is all trans retinoic acid

13. The method of claim 9 wherein said cyclin dependent kinase inhibitor is selected from the group consisting of flavopiridol, UCN-01, roscovitine, olomoucine, and butyrolactone.

14. The method of claim 8 wherein said cancer cells are selected from the group consisting of leukemia cells, prostate cancer cells, breast cancer cells, myeloma cells, and lymphoma cells.

15. A composition comprising,
a cyclin dependent kinase inhibitor,
an agent that induces cell differentiation, and
a carrier suitable for in vivo administration.

16. The method of claim 15 wherein said agent that induces cell differentiation is selected from the group consisting of histone deacetylation inhibitors, protein kinase C activators, retinoids, and Vitamin D3.

17. The method of claim 16 wherein said histone deacetylation inhibitor is selected from the group consisting of sodium butyrate, phenylbutyrate, suberoylanilide hydroxamic acid, depsipeptide, tricostatin A, MS-275, and CI-994.

18. The method of claim 16 wherein said protein kinase C activator is selected from the group consisting of PMA and bryostatin.

19. The method of claim 16 wherein said retinoid is all trans retinoic acid.

20. The method of claim 16 wherein said cyclin dependent kinase inhibitor is selected from the group consisting of flavopiridol, UCN-01, roscovitine, olomoucine, and butyrolactone.
